(19) United States
(12) Patent Application Publication
(10) Pub. No.: US 2019/0069989 A1
(43) Pub. Date: Mar. 7, 2019

(54) Multipart electrowetting intraocular lens for in-situ assembly

(71) Applicant: Verily Life Sciences LLC, Mountain View, CA (US)

(72) Inventors: Daniel Otts, Pleasanton, CA (US); Stein Kuiper, South San Francisco, CA (US)

(21) Appl. No.: 16/109,403

(22) Filed: Aug. 22, 2018

Related U.S. Application Data
(60) Provisional application No. 62/554,958, filed on Sep. 6, 2017.

(51) Int. Cl. A61F 2/16

(52) U.S. Cl. CPC A61F 2/1648; A61F 2002/482; A61F 2/1627

(57) ABSTRACT

An eye-implantable electrowetting lens can be operated to control an overall optical power of an eye in which the device is implanted. A lens chamber of the electrowetting lens contains first and second fluids that are immiscible with each other and have different refractive indexes. By applying a voltage to electrodes of the lens, the optical power of the lens can be controlled by affecting the geometry of the interface between the fluids. To facilitate implantation of such an eye-implantable device, components of the device may be inserted individually and the device may be assembled in situ. In situ assembly could allow insertion of components of the device into the eye through an incision that is smaller than the assembled device, reduce a chance of failure of the device, reduce the mechanical and/or chemical seal requirements of the assembled, or allow for modular device design.

### CROSS-REFERENCE TO RELATED APPLICATION

This application claims priority to U.S. Provisional Patent Application No. 62/554,958, filed Sep. 6, 2017, which is incorporated herein by reference

### BACKGROUND

Unless otherwise indicated herein, the materials described in this section are not prior art to the claims in this application and are not admitted to be prior art by inclusion in this section.

Devices can be provided on the surface of the eye and/or within the eye to provide a variety of functions. In some examples, these functions can include functions to improve the ability of a person to view their environment (e.g., to provide an optical correction, to stimulate the retina directly) and/or to present additional visual information to the person (e.g., to present a heads up display or other indications to the person). Additionally or alternatively, these functions can include detecting a property of the body of a person (e.g., a blood glucose level, a concentration of an ion in the blood, a desired optical power of the eye) via the eye, e.g., by detecting forces, concentrations of analytes, electrical fields, or other properties related to the property of interest. Such functions can be provided by an intraocular device implanted within the eye (e.g., a retinal implant configured to stimulate the retina to restore vision, a device implanted within the lens capsule to provide a static and/or controllable optical power to the eye).

### SUMMARY

Some embodiments of the present disclosure provide an eye-implantable device that may be assembled in situ. The eye-implantable device includes: (i) a first chamber portion comprising a first mating surface, wherein the first chamber portion is flexible; (ii) a second chamber portion comprising a second mating surface, wherein the second mating surface is shaped to mate with the first mating surface such that the first chamber portion and the second chamber portion form a lens chamber of an electrowetting lens, and wherein the second chamber portion is flexible; (iii) a fluid configured to be inserted into the lens chamber of the electrowetting lens formed by the first chamber portion and the second chamber portion onto a surface of at least one of the first chamber portion or the second chamber portion.

Some embodiments of the present disclosure provide a method comprising: (i) forming an incision through a cornea of an eye; (ii) inserting a first chamber portion into the eye through the incision, wherein the first chamber portion comprises a first mating surface, and wherein the first chamber portion is flexible; (iii) inserting a second chamber portion into the eye through the incision, wherein the second chamber portion comprises a second mating surface that is shaped to mate with the first mating surface, wherein the second chamber portion is flexible, wherein the second chamber portion comprises an electrode, and wherein the electrode comprises a dielectric layer; (iv) introducing a fluid into the eye such that the fluid is disposed on a surface of the second chamber portion in contact with the second electrode; and (v) forming a lens chamber of an electrowetting lens by coupling the first chamber portion to the second chamber portion, wherein coupling the first chamber portion to the second chamber portion comprises mating the first mating surface with the second mating surface.

Some embodiments of the present disclosure provide for an eye-implantable device comprising an electrowetting lens. The electrowetting lens comprises: (i) a first chamber portion comprising a first mating surface, wherein the first chamber portion is flexible; (ii) a second chamber portion comprising a second mating surface, wherein the second chamber portion is flexible, and wherein the first mating surface is mated with the second mating surface such that the first chamber portion and the second chamber portions form a lens chamber of the electrowetting lens; (iii) a first fluid disposed in the lens chamber; (iv) a second fluid disposed in the lens chamber, wherein the second fluid is immiscible with the first fluid, and wherein a refractive index of the second fluid differs from a refractive index of the first fluid; (v) a first electrode, wherein the first electrode is in contact with the first fluid; and (vi) a second electrode, wherein the second electrode is in contact with at least one of the first fluid or the second fluid, and wherein the second electrode comprises a dielectric coating.

Some embodiments of the present disclosure also provide for a component for an eye-implantable device comprising: (i) a chamber portion comprising a concave lens chamber surface, wherein the chamber portion is flexible; (ii) a first electrode disposed on the concave lens chamber surface; (iii) a second electrode disposed on the concave lens chamber surface, wherein the second electrode comprises a dielectric coating; and (iv) a controller, wherein the controller is electronically coupled to the first electrode and the second electrode, and wherein the controller is configured to apply a voltage between the first electrode and the second electrode.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A is a perspective view of an example eye-implantable device.

FIG. 1B is a side cross-section view of an electrowetting lens of the example eye-implantable device shown in FIG. 1A.

FIG. 1C is a perspective view of a first component of an example eye-implantable device.

FIG. 1D is a perspective view of a second component of an example eye-implantable device.

FIG. 1E is a perspective view of an example eye-implantable device comprising the components shown in FIGS. 1C and 1D.

FIG. 1F is a side cross-section view of the example eye-implantable device shown in FIG. 1E located within an eye.

FIG. 2A is a side cross-section view of an example eye-implantable device.

FIG. 2B is another side cross-section view of the example eye-implantable device shown in FIG. 2A.

FIG. 3A is a side cross-section view of a chamber portion of an example eye-implantable device.

FIG. 3B is a side cross-section view of the chamber portion shown in FIG. 3A with a fluid disposed thereon.

FIG. 3C is a side cross-section view of a further chamber portion of an electrowetting lens.

FIG. 3D is a side cross-section view of the example chamber portion shown in FIG. 3B after the placement of the further chamber portion shown in FIG. 3C thereon.

FIG. 3E is another side cross-section view of the example chamber portions of an eye-implantable device shown in FIG. 3C-D.

FIG. 4A is a side cross-section view of an example eye-implantable device.

FIG. 4B is a side cross-section view of an example eye-implantable device.

FIG. 4C is a side cross-section view of an example eye-implantable device.

FIG. 4D is a side cross-section view of an example eye-implantable device.

FIG. 4E is a side cross-section view of an example eye-implantable device.

FIG. 5 is a block diagram of an example system that includes an extraocular device and an eye-implanted device.

FIG. 6 is a flowchart of an example process.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### I. OVERVIEW

Implantable devices could be located within an eye of a person to provide a static or adjustable optical power to the eye. Such a static or adjustable optical power could be provided to correct a lack or loss of optical power and/or accommodation in the eye, e.g., to correct for presbyopia, myopia, hyperopia, astigmatism, injury or damage to the eye, removal of the crystalline lens of the eye, or to correct for some other condition of the eye. Such implantable devices could be located within the lens capsule, within the anterior chamber, within the fibrous wall of the eye, proximate to the retina, or in some other location(s) of the eye according to an application. Such an eye-implantable device could include an electronically actuated lens to provide a controllable amount of optical power to the eye. An electronically actuated lens could include an electrowetting lens that includes two or more immiscible fluids whose geometry within the electrowetting lens can be electronically controlled (e.g., by applying an electrical voltage to two or more electrodes of the lens) in order to control an overall optical power of the electrowetting lens.

To facilitate implantation of such an eye-implantable device, it can be beneficial for the device to be assembled in situ. In situ assembly could allow insertion of components of the device into the eye through an incision that is smaller than the assembled device and/or than a characteristic size of the assembled device when rolled, folded, or otherwise configured to facilitate such insertion. In situ assembly may also reduce a chance of failure of the device or otherwise improve operation of the device following implantation. For example, components of the electrowetting lens could be inserted into the eye without one or more immiscible fluids of the electrowetting lens. These fluids could be added later, preventing the fluids from mixing or otherwise dispersing into each other, from contacting and wetting certain surfaces within the assembled electrowetting lens, or from producing other deleterious effects that could affect the optical properties or functioning of the assembled electrowetting lens. In situ assembly may reduce the mechanical and/or chemical seal requirements of the assembled device relative to a device that is not assembled in situ and that is inserted as a pre-assembled, fluid-filled electrowetting lens. In situ assembly may also allow for modular device design, such that individual device components may be specified and/or selected according to a particular user (e.g., based on a specified optical power, size, fluid volume, coloration, or other consideration of the particular user).

In situ assembly of such a device could include individually inserting, positioning, or otherwise manipulating two or more components of the device prior to assembling the components to form the complete device. For example, a first component that includes a first portion of a lens chamber of an electrowetting lens can be positioned within the lens capsule of the eye. Further components and/or fluids of the device may subsequently be disposed on or within, mounted or adhered to, or otherwise added to the first component to assemble the eye-implantable device. Assembly could include introducing fluids of the electrowetting lens, electronic components, and/or further portion(s) of the lens chamber. For example, an additional component, comprising a window of the electrowetting lens, may be coupled to the first component to create a sealed lens chamber of the electrowetting lens. Such an electrowetting lens could then be operated to provide an electronically-controllable optical power.

Elements of such an eye-implantable device could be distributed amongst multiple different components, which may be assembled in situ in a variety of ways. In some examples, a first component could include two or more electrodes, a controller coupled to the electrodes, and other electronic components to facilitate functioning of the electrowetting lens. Further components could lack electrodes or other electronic components and could function to mechanically seal the electrowetting lens chamber and/or to provide an optical power. Additionally or alternatively, one or more electrodes and/or other electronic components may be included in a further component of such an eye-implantable device. Such a further component and/or the first component could include contacts, pins, penetrating elements, or other components to facilitate electronic coupling between electrodes, controller(s), and/or other electronic elements when such components are assembled to form the eye-implantable device. In some embodiments, a controller or other electronic components may be electronically coupled to the electrodes of an electrowetting lens of such an eye-implantable device via a cable or tether.

Two or more components of such an eye-implantable device, comprising respective chamber portions of a lens chamber, can be mated along respective mating surfaces to form the lens chamber of the eye-implantable device. The first and second chamber portions may be mated through a press or snap fit. Additionally or alternatively, the chamber portions may be coupled together using a pressure-sensitive adhesive disposed on the mating surface of one or more of the lens chamber portions. The first and second chamber portions may additionally or alternatively be coupled using heat sealing (e.g., localized application of heat), a clamp, by applying a curable sealant, or through some other means to secure the components of the assembled lens chamber together and/or to provide a seal to inhibit fluid flow into or out of the formed lens chamber.

In situ assembly of an eye-implantable device as described herein could include introducing one or more fluids into the eye. Such fluids could be immiscible and differ with respect to refractive index (e.g., saline or another aqueous fluid and oil or another non-polar fluid) such that a surface of contact between the fluids can provide an optical power. Such fluids may be introduced after the insertion of at least one component of a lens chamber of an electrowetting lens of the eye-implantable device, but before the assembly and/or sealing of the lens chamber of the electrowetting lens. For example, an oil or other nonpolar fluid could be deposited onto or inside a first component of the eye-implantable device after the first component has been inserted into the eye. The oil could be deposited onto a surface of the first component that provides a first internal surface of a lens chamber of the electrowetting lens. Subsequent assembly of the first component with a second component can form the lens chamber of the electrowetting lens such that the oil, along with an amount of a second fluid (e.g., an amount of a saline fluid introduced into the eye, an amount of aqueous humor of the eye), is disposed within the assembled electrowetting lens. In other examples, one or more fluids may be introduced into the lens chamber of the electrowetting lens after the lens chamber has been formed. For instance, an oil may be disposed onto a surface of a lens chamber after two or more components have been inserted, unfolded/unrolled, mated together, or otherwise manipulated to form the lens chamber. Additionally or alternatively, one or more fluids may be removed from such a formed lens chamber (e.g., to maintain a total volume of fluid within the lens chamber at a specified level, to rinse bubbles out of the lens chamber, or to rinse debris, aqueous humor, or other substances out of the eye).

In situ assembly of an eye-implantable device as described herein may also include washing, irrigation, and/or debubbling of a region within an eye (e.g., the lens capsule of the eye) to remove debris, clear excess viscoelastic gels applied during a surgical procedure, improve the optical clarity of the assembled device, or to provide some other benefit. Verification testing can also be performed to determine whether the assembled device is functional, to adjust individual components, or to calibrate the device (e.g., to determine the optical power provided by the device as a function of, e.g., a voltage applied between electrodes of the device). This could include applying a voltage between the electrodes of the device to assess the functionality and optical performance of the assembled electrowetting lens. Other methods of in situ assembly, manipulation, and/or testing of eye-implantable device components as described herein are contemplated.

Elements of such an eye-implantable device could be flexible. Such flexible components could be bent, folded, or otherwise manipulated to permit implantation. The components could subsequently be unfolded or otherwise manipulated into a flat or otherwise operational state prior to the assembly of the electrowetting lens device. For example, the eye-implantable device could be rolled up or folded (e.g., in half, in thirds) to facilitate insertion into the eye by way of an incision that is smaller than the unfolded size of the device (e.g., via an incision that is smaller than an unfolded diameter of an electrowetting lens of the eye-implantable device). Such flexibility could improve biocompatibility, speed or otherwise improve the process of implantation, permit detection of forces applied to the device, or could provide some other benefit.

Such eye-implantable devices could further include electronics, antennas, voltage regulators, batteries, photovoltaic cells, sensors, or other elements to facilitate operations of the device, e.g., to provide a controllable optical power to an eye. Such eye-implantable devices could receive, from outside of the eye, radio frequency, optical, infrared, acoustic, or other forms of power to power the operations of the device, e.g., from a contact lens, eyeglasses, a head-mountable device, or some other source. The eye-implantable device could receive wireless transmissions to specify an amount of optical power to provide, via controlling the optical power of the electrowetting lens, to the eye, could operate a sensor to detect a physical variable (e.g., an accommodation force exerted by ciliary muscles of the eye) to specify the amount of optical power to provide, or the eye-implantable device could use some additional or alternative source of information or commands to determine an amount of optical power to provide to an eye.

### II. EXAMPLE EYE-IMPLANTABLE DEVICE

An eye-implantable device (e.g., an intraocular lens, or IOL) can include electronics and an electronically actuated lens that are operable to provide a controllable optical power (e.g., a controllable diopter, focal length, or other form of optical power or refractive property) to an eye in which the device is implanted. Such an eye-implantable device may be assembled in situ from two or more components; such components could include respective portions of a lens chamber of an electrowetting lens, two or more electrodes, and at least one fluid disposed on a surface within a lens chamber. Device components could include haptics or other formed features or be formed according to a particular shape such that the assembled eye-implantable device can be implanted in or at a particular location within an eye. Such a location within the eye could include the lens capsule of the eye following removal of the crystalline lens, the anterior chamber of the eye, the posterior chamber of the eye, and/or along an optical axis of the eye. A controller, battery, antenna, sensors, or other elements can be provided to power the device, to determine a specified amount of optical power to provide to the eye (e.g., based on a sensor output, based on a received wireless command), and to operate the electronically actuated lens to provide such a specified optical power by applying a voltage, current, or other electrical signal to the electronically actuated lens. In some examples, the electronically actuated lens could be an electrowetting lens.

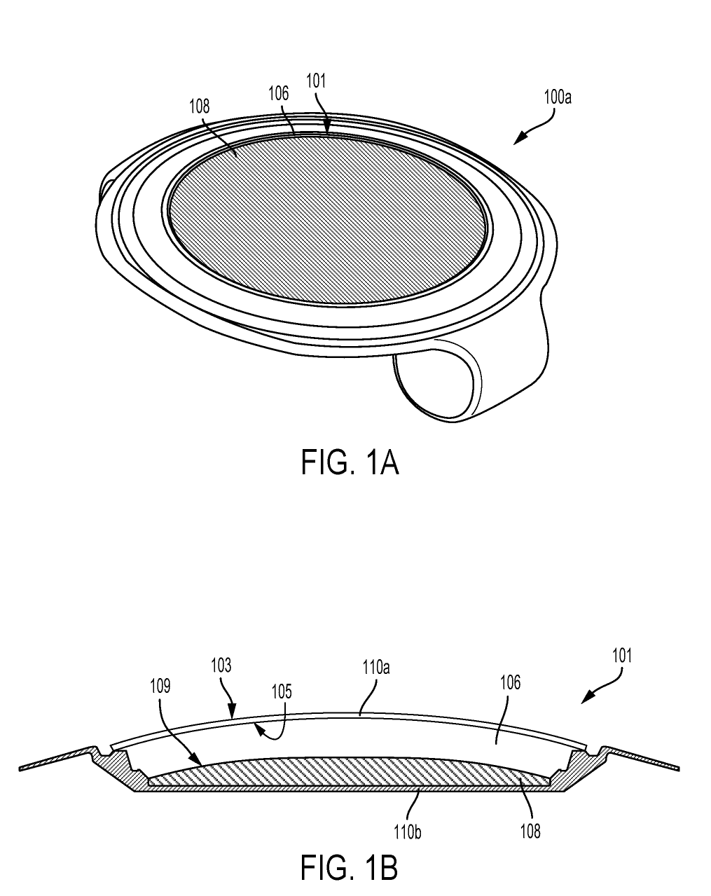

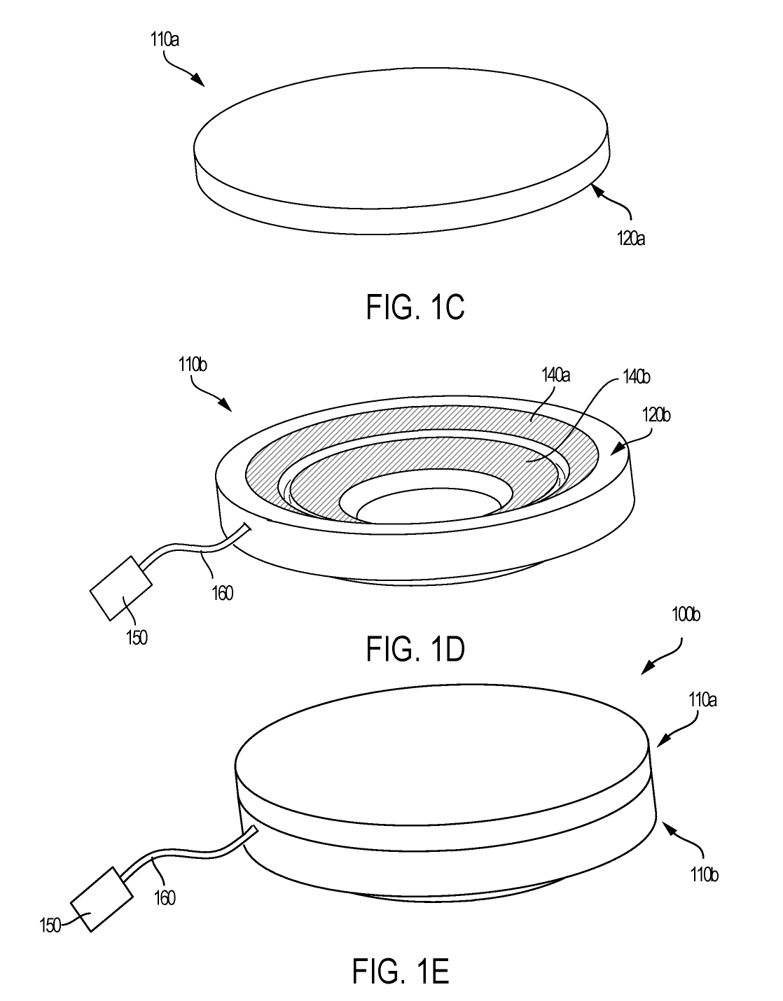

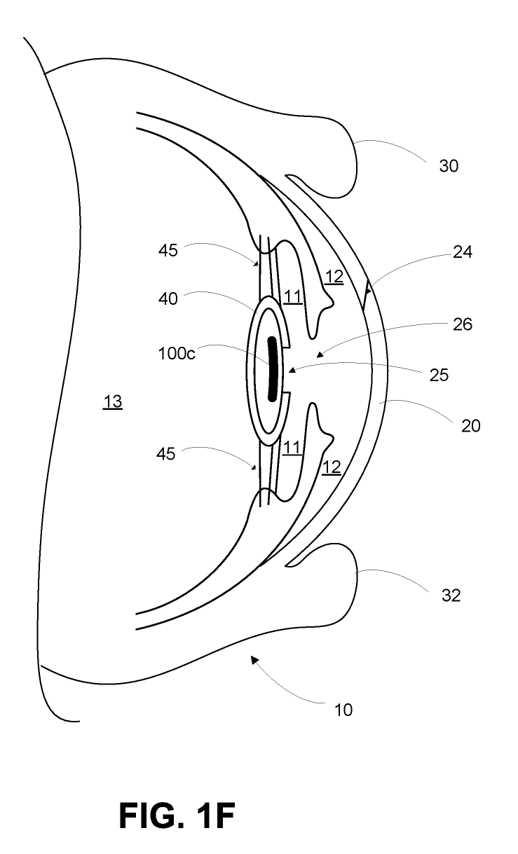

FIG. 1A is a bottom view of an example eye-implantable device 100a. FIG. 1B is a cross-sectional view of an electrowetting lens 101 of the example eye-implantable device 100a shown in FIG. 1A. It is noted that relative dimensions in FIGS. 1A and 1B are not necessarily to scale, but have been rendered for purposes of explanation only in describing the arrangement of the example eye-implantable device 100a and electrowetting lens 101 thereof.

The eye-implantable device 100a includes electronics (not shown) configured to operate the electrowetting lens 101 to provide a controllable optical power and to provide other operations of the eye-implantable device 100a. The electronics may include electrodes, controllers, voltage regulators, antennas, photovoltaic cells, sensors, transmitters, receivers, batteries, or other components. Such electronics may be embedded within the illustrated elements of the eye-implantable device 100a (e.g., embedded within a polymeric material that forms part the device shown in FIG. 1A) or disposed on a surface of the device. In one embodiment, a battery and/or other electronics could be mounted on or within a material that forms a lens chamber or some other portion of the electrowetting lens 101. Additionally or alternatively, such electronics could be provided external to the eye-implantable device. For example, electronics could be provided in an additional device that is implantable in a sclera or lens capsule of the eye. In such cases, the electronics may be coupled to device 100a via a wired connection (e.g., via a tether) or a wireless connection. The eye-implantable device could additionally include haptics or other formed elements to maintain the electronics at a particular location within the eye (e.g., within a lens capsule of an eye) or to provide some other benefit.

The electronics may be configured to receive and/or store wireless energy to power the device 100a (e.g., visible light energy, infrared light energy, radio frequency electromagnetic energy, acoustic energy), to communicate with external devices or systems (e.g., to receive program updates, to receive a commanded optical power level), to detect one or more physical variables (e.g., a light level, a pupil diameter, an intraocular pressure, a voltage related to activity of muscles of the eye, a force exerted by ciliary muscles of the eye, a concentration of one or more substances in the eye) that may be used to determine an optical power to provide or that may be used in some other way, to operate the electrowetting lens 101, or to facilitate some other applications of the device 100a.

Components of the electrowetting lens 101 and/or other elements of the eye-implantable device 100a may be formed of one or more polymeric materials. The polymeric materials can include substantially transparent materials to allow incident light to be transmitted to the retina of the eye through the electrowetting lens 101 of the eye-implantable device 100a. The polymeric materials can include biocompatible materials similar to those employed to form implants, vision correction lenses, IOLs, or other implantable devices, such as polyethylene terephthalate (“PET”), polymethyl methacrylate (“PMMA”), silicone, silicone hydrogels, rigid, gas-permeable polymeric materials, barrier materials that block diffusion of gases or other substances, combinations of these, etc. The polymeric materials could include flexible and/or foldable water-permeable materials. For example, the polymeric material could include a copolymer comprising 2-phenylethyl acrylate units and 2-phenylethyl methacrylate units. Units of a polymer or copolymer could be cross-linked by an applicable cross-linking agent or unit, e.g., by 1,4-butanediol diacrylate units, 1,6-hexanediol diacrylate units, or some other crosslinking agent or combination of such agents.

Such flexible and/or foldable materials may be included in the construction of the device 100a (e.g., in the construction of individual components that may be assembled, in situ, to form the device) to permit the device 100a (or components thereof) to be rolled, folded, or otherwise manipulated such that the device 100a may be inserted through an incision that is smaller than, e.g., the diameter of the unrolled or un-folded electrowetting lens 101 or the diameter of an unrolled or un-folded component of the electrowetting lens 101. The eye-implantable device 100a may include coating materials disposed on one or more external or internal surfaces of the device, e.g., to improve a biocompatibility of the device, to control a surface energy of an internal surface of the electrowetting lens (e.g., to encourage or prevent wetting of a surface within a lens chamber by one or more fluids within the lens chamber), to prevent passage of ions or other substances, or to provide some other benefit.

The electrowetting lens 101 includes a first fluid 106 and a second fluid 108 disposed on an internal surface of the lens chamber in contact with the first and/or second electrodes (not shown). The first 106 and second 108 fluids are immiscible (e.g., the first fluid 106 could be saline or some other aqueous fluid and the second fluid 108 could be an oil or some other nonpolar fluid) and differ with respect to refractive index. Thus, a surface of contact 109 between the first 106 and second 108 fluids (e.g., a convex shape, as shown in FIG. 1B) could provide an optical power (e.g., a diopter, a finite, nonzero focal length) related to the difference in the refractive indices of the fluids 106, 108 and the shape of the surface of contact 109.

One of the first 106 or second 108 fluid may include an aqueous solution. Such an aqueous solution may be electrically conductive, e.g., to facilitate transmission of electrical voltages or currents through the aqueous solution in order to control the shape of the interface between the aqueous solution and another fluid of the electrowetting lens 101.

The overall optical power provided by the eye-implantable device 100a and/or the electrowetting lens 101 (e.g., to an eye in which the device 100a is implanted) could be related to the geometry, refractive index, or other properties of elements of the eye-implantable device 100a. This could include the shape of a contact surface 109 between the first 106 and second 108 fluids within the lens chamber and the refractive indices of the fluids 106, 108. A static optical power may also be provided by the curvature of one or more components of the electrowetting lens 101. For example, one or more of the chamber portions may comprise a convex optical surface 103 and/or a concave lens surface 105, and at least one of the convex optical surface or the concave lens surface may have a curvature specified to provide a base optical power to the user.

An eye-implantable device (e.g., 100a) could be composed of multiple components which may be inserted separately and assembled in situ. Such an eye-implantable device may be assembled in situ from two or more components; such components could include respective portions of a lens chamber of an electrowetting lens, two or more electrodes, and at least one fluid disposed on a surface within a lens chamber. In some examples, two or more components are assembled together to form respective portions of the electrowetting lens 101 of device 100a. For example, first 110a and second 110b elements comprising electronics, lenses, fluids, and/or other components may be mated within the eye to form the electrowetting lens 101 of the eye-implantable device 100a.

FIG. 1C shows a perspective view of a first chamber portion 110a of an electrowetting lens of an eye-implantable device. Such a first chamber portion 110a may be roughly in the form of a curved transparent polymer disk, with a first mating surface 120a shaped to interact with a corresponding second mating surface of a further chamber portion of the eye-implantable device. The first chamber portion could comprise a transparent polymeric material that is biocompatible. For example, the polymeric material could include a copolymer comprising 2-phenylethyl acrylate units and 2-phenylethyl methacrylate units, a silicone, a hydrogel, or some other material or combination of materials. The first chamber portion 110a could also include flexible and/or foldable materials to permit rolling, folding, or other manipulations to facilitate insertion of the first chamber portion 110a through an incision that is smaller than, e.g., the diameter of the unrolled or un-folded electrowetting lens or the diameter of an unrolled or un-folded first chamber portion 110a of the electrowetting lens.

FIG. 1D illustrates a perspective view of a second chamber portion 110b, which may be mated with the first chamber portion 110a to form the electrowetting lens of the eye-implantable device. The second chamber portion 110b could also be roughly in the form of a transparent polymer disk. Such a second chamber portion 110b may comprise a flexible material, which could permit rolling, folding, or other manipulations to facilitate insertion of the second chamber portion into the eye. The second chamber portion includes a second mating surface 120b that is shaped to mate with the first mating surface 120a such that such that the first chamber portion 110a and the second chamber portion 110b form the lens chamber of the electrowetting lens 110 when the first mating surface 120a is mated with (e.g., placed into contact with) the second mating surface 120b. The second chamber portion additionally includes a first electrode 140a, a second electrode 140b, a controller 150, and a tether 160. The first 110a and/or second 110b chamber portions may include additional or alternative components, mating or otherwise configured surfaces, or other features or elements to facilitate the assembly and/or operation of the electrowetting lens.

Elements of the electrowetting lens 110 (e.g., electrodes, controllers, antennas, sensors, other electronic components, etc.) could be distributed amongst the first and second chamber portions in a variety of ways. For example, a first and/or second electrode 140a, 140b could be disposed on the concave lens chamber surface of at least one of the first chamber portion or second chamber portion. As illustrated in FIGS. 1C-D, in some examples the second lens chamber portion 110b could include a first electrode 140a and a second electrode 140b in the form of two concentric inclined rings. The first electrode 140a and second electrode 140b may be coupled such that a voltage can be applied between them to operate the electrowetting lens 110. One or both of the electrodes could further include a dielectric layer disposed between the electrode surface and the inside of the lens chamber 110. Additionally or alternatively, one or more electrodes and/or other electronic components may be included in the first chamber portion 110a of such an eye-implantable device 100. For example, the first chamber portion 110a could comprise a first electrode 140a, the second chamber portion 110b could comprise a controller 150 and a second electrode 140b electronically coupled to the controller 150, and the controller could be electronically coupled to the first electrode when the first mating surface is mated with the second mating surface. The first or second chamber portions 110a, 110b could also include contacts, pins, penetrating elements, or other components to facilitate electronic coupling between electrodes, controller(s), and/or other electronic elements when the first and second chamber portions are assembled into the eye-implantable device 100.

The first 110a or second 110b lens chamber portions may further comprise a controller 150 which is operable to apply a voltage across the first 140a and second 140b electrodes. A geometry of interface 131 between the first fluid and second fluid could be related to the voltage applied by the controller 150 between the first 140a and second 140b electrodes, and the optical power of the electrowetting lens 110 could be related to the geometry of the interface 131 between the first 130a and second 130b fluids. In some embodiments, the controller 150 could be embedded within one or both of the first or second chamber portions 110a, 110b or located on a surface of one or both of the chamber portions. In another embodiment, the controller 150 may be located external to the electrowetting lens 110 and may be coupled to the second chamber portion 110b and/or interfaced with other electronic components via a tether 160. Additionally or alternatively, the controller could be interfaced with electronic components of the chamber portion(s) via a wireless connection.

FIG. 1E illustrates a perspective view of the eye-implantable device (shown as 110b) formed from the first chamber portion 110a and the second chamber portion 110b when the first and second mating surfaces are mated together. Such mating of the mating surfaces forms an enclosed lens chamber of the electrowetting lens of the eye-implantable device 100b such that the first electrode 140a and the second electrode 140b are disposed on respective internal surfaces of the lens chamber of the electrowetting lens. It is noted that relative dimensions in FIGS. 1C, 1D, and 1E are not necessarily to scale, but have been rendered for purposes of explanation only in describing the arrangement of the example eye-implantable device 100b and chamber portions 110a, 110b thereof.

Mating of the first 110a and second 110b chamber portions could include one or more of a press or snap fit, adhesion of the chamber portions together by a pressure-sensitive adhesive that is disposed on one or both of the mating surfaces, adhesion of the chamber portions through the use of a self-healing polymer. applying heat to melt a meltable adhesive material of one or both of the chamber portions, applying a clamping component to clamp the chamber portions together, applying a curable sealant to one or both of the chamber portions, or using some other method or means to mate the mating surfaces together, to mechanically couple the chamber portions together, to form a seal between the chamber portions (e.g., to inhibit fluid flow into or out of the lens chamber), or to facilitate some other application.

Note that the illustrated first 110a and second 110b chamber portions of the eye-implantable device 100b are intended as non-limiting example embodiments of components that may be assembled together to form an eye-implantable device as described herein. For example, an electrowetting lens and/or a lens chamber thereof as described herein could be constructed from more or fewer components (e.g., from a front element, a rear element, and an annular element) than the two shown in FIGS. 1C-E and/or could be constructed from components configured differently from the chamber portions 110a, 110b illustrated here. Different components of the eye-implantable device 100b could be composed of the same material (e.g., the first and second chamber portions 110a, 110b of the eye-implantable device 100b could both be composed of a copolymer comprising 2-phenylethyl acrylate units and 2-phenylethyl methacrylate units). Alternatively, elements of an electrowetting lens could be composed of respective different materials (e.g., the first chamber portion 110a could be composed of a copolymer comprising 2-phenylethyl acrylate units and 2-phenylethyl methacrylate units and the second chamber portion 110b could be composed of polyethylene terephthalate). At least a portion the first 110a or second 110b chamber portions may include a surface coating. For example, the concave lens chamber surface of the first 110a and/or second 110b chamber portion may comprise a hydrophobic coating to facilitate wetting by an oil, a hydrophilic coating to facilitate wetting by an aqueous solution, an underwater oleophobic coating to prevent wetting by an oil when submerged in an aqueous solution, or some other coatings or surface treatments.

At least a portion of the first 110a and/or second 110b chamber portions of the electrowetting lens 110 could be formed from a polymeric material (e.g., one of the polymeric materials listed elsewhere herein) that is permeable to water in aqueous humor of an eye (e.g., from a copolymer comprising 2-phenylethyl acrylate units and 2-phenylethyl methacrylate units cross-linked by 1,4-butanediol diacrylate units). Such a water-permeable polymeric material, or other polymeric or non-polymeric materials of the eye-implantable device 100b, could be flexible such that the chamber portions 110a, 110b can be rolled, folded, or otherwise manipulated, e.g., to facilitate insertion through an incision in an eye. Additionally or alternatively, one or more sealant materials (e.g., a sealant material used to adhere the first chamber portion 110a to the second chamber portion 110b) of the eye-implantable device 100b could be permeable to water in aqueous humor of an eye.

The eye-implantable device 100b includes first and second fluids (not shown) disposed on internal surfaces of the electrowetting lens chamber in contact with the first 140a and/or second 140b electrodes. In some embodiments, the first and second fluids are two immiscible fluids that differ with respect to refractive index. For example, the first fluid could comprise a saline or another polar fluid, while the second fluid could comprise an oil or another non-polar fluid. Thus, a surface of contact between the first and second fluids could provide an optical power (e.g., a diopter, a nonzero focal length) related to the difference in the refractive indices of the fluids and the shape of the surface of contact.

Such fluids could be introduced into an eye after at least one of the first 110a or second 110b chamber portions has been inserted, unrolled, flattened, manipulated to assume a specified shape and/or manipulated to occupy a specified location within the eye (e.g., a location within the lens capsule of the eye). In some embodiments, a first fluid (e.g., an oil) may be deposited on a surface of the second chamber portion prior to mating of the first 110a and second 110b chamber portions to form the eye-implantable device 100b (e.g., to form and enclose the lens chamber of the eye-implantable device 100b). Additionally or alternatively, the first and/or second fluids may be introduced into the lens chamber of the eye-implantable device 100b after the eye-implantable device 100b has been formed. For example, one or more fluids may be disposed in a lens chamber after the first 110a and second 110b chamber portions have been inserted, unfolded/unrolled, mated together, or otherwise manipulated to form the lens chamber. This could include introducing the one or more fluids via one or more tubes that are in fluid communication with the lens chamber (e.g., tubes that formed part of one of the chamber portions 110a, 110b), via a needle piercing a septum or other feature of the chamber portions 110a, 110b, or via some other method.

The first electrode 140a and a second electrode 140b are disposed on respective surfaces of the second lens chamber portion 110b which, when the second chamber portion 110b is mated to the first chamber portion 110a, form internal surfaces of the lens chamber of the formed eye-implantable device 100b. In order to allow the chamber portions 110a, 110b to be flexed, folded, rolled, or otherwise manipulated during implantation while retaining the functionality of the eye-implantable device 100b, the electrodes could be composed of gold, aluminum, silver nanowires, or some other material or coating that can be flexed and maintain an overall level of electrical conductivity across the area of the electrodes. Such materials could be applied mechanically (e.g., as a foil) or via some other process (e.g., via sputtering, CVD, PVD, application as a solution followed by evaporation of a solvent of the solution).

Voltages, currents, or other electrical signals can be applied to the at least two electrodes 140a, 140b to electronically control the shape of first and second fluids (e.g., to control a shape of a contact surface between the two fluids) in order to control an optical power of the electrowetting lens of the eye-implantable device 100b. The overall optical power provided by the eye-implantable device 100b and/or the electrowetting lens thereof (e.g., to an eye in which the device 100b is implanted) could be related to the geometry, refractive index, or other properties of elements of the eye-implantable device 100b. This could include the shape of a contact surface between the first and second fluids within the lens chamber and the refractive indices of the fluids. Other elements of the eye-implantable device 100b could also provide a static and/or controllable optical power. For example, the first 110a or second 110b chamber portions of the eye-implantable device 100b could have curved surfaces (e.g., a curved convex optical surface) specified to provide an optical power related to a change in refractive index between materials on either side of those surfaces (e.g., between a polymeric material of the first 110a and/or second 110b chamber portions and aqueous humor of an eye, or between the polymeric material and one of the first or second fluids within the lens chamber of the eye-implantable device 100b).

Components of the eye-implantable device 100b (e.g., the first 110a or second 110b elements forming the eye-implantable device 100b) can be formed to have a curved shape in a variety of ways. For example, techniques similar to those employed to form vision-correction contact lenses and/or intraocular lenses, such as heat molding, injection molding, spin casting, etc. can be employed to form polymeric materials into components of the eye-implantable device 100b. Further, an eye-implantable device as described herein could have a different shape from that of the illustrated eye-implantable device components 100a, 100b. For example, an eye-implantable device could include haptics or other formed elements to maintain the eye-implantable device at a particular location within an eye (e.g., within a lens capsule of an eye), to detect accommodation forces exerted by ciliary muscles of an eye, or to provide some other benefit.

In some embodiments, properties of one or more of the components of such an eye-implantable device 100b can be adjusted according to a particular patient (e.g., based on a specified optical power, size, thickness, coloration, curvature, or other considerations related to the particular patient). Such adjustment may include modifying or customizing a component to have a specified property (e.g., by machining, by adding or removing material, by heat-forming a component). Additionally or alternatively, this adjustment could include selecting the component from a set of components that vary with respect to one or more properties (e.g., curvature, size, optical power). In some embodiments, an interface between two or more of the components comprising the eye-implantable device 100b may be standardized such that one or more of the components may be interchanged to adjust the properties (e.g., to adjust a baseline optical power) of the eye-implantable device assembled therefrom. For example, an interface (e.g., a shape of alignment features, clamping features, snap-fit features, or other elements of a mating surface) between the first component 110a and the second component 110b may be standardized. This could include a shape or arrangement of alignment features, snap-fit features, or other formed features or elements of corresponding mounting surfaces of the first 110a and second 110b components conforming to a standard. In such examples, the first and/or second components may be selected from respective sets of alternative components in order to specify one or more properties (e.g., baseline optical power, device diameter, device color) of the eye-implantable device formed when the components 110a, 110b are mated together. Such a formed eye-implantable device may have properties that are patient-specific and dependent on the properties of the selected first and/or second components.

FIG. 1F is a side cross-section view of an eye-implantable device 100c (e.g., 100a, 100b) while implanted within an eye 10. The eye 10 includes a cornea 20 that is covered by bringing the upper eyelid 30 and lower eyelid 32 together over the top of the eye 10. Incident light is received by the eye 10 through the cornea 20, where light is optically directed to light sensing elements of the eye 10 (e.g., rods and cones, etc.) to stimulate visual perception.

The light received by the retina is transmitted, in the unaltered eye, through the crystalline lens, being refracted by the lens such that light received from the environment arrives in focus at the retina. The crystalline lens is located within the lens capsule 40 of the eye, which is connected, via the zonules 45, to accommodation muscles (e.g., ciliary muscles) and other elements of the eye. Accommodation forces transmitted through the zonules (e.g., forces generated by the accommodation muscles, forces generated by intrinsic elasticity of the zonules, or forces generated by other sources) act, in the eye, to deform the crystalline lens within the lens capsule 40, controlling the optical power provided by the crystalline lens.

As shown in FIG. 1F, the crystalline lens of the eye 10 has been removed and the eye-implantable device 100c has been surgically emplaced within the lens capsule 40 such that light received by the retina is transmitted through an electrowetting lens of the eye-implantable device 100c, being refracted by the electrowetting lens and/or other elements of the eye-implantable device 100c. Thus, the eye-implantable device 100c can be operated such that light received from the environment may arrive in focus at the retina, e.g., by operating the electrowetting lens to provide a specified optical power.

Components of the eye-implantable device 100c (e.g., 110a, 110b) have been inserted into the eye 10 through an incision 24 formed in the cornea 20 of the eye 10, assembled to form the eye-implantable device 100c, and positioned within the lens capsule 40. In order to position the device 100c within the lens capsule 40, a hole 25 has been formed in the lens capsule 40 (e.g., via continuous curvilinear capsulorhexis) and the crystalline lens has been removed (e.g., via ultrasonic phacoemulsification). An eye-implantable device as described herein may be positioned in alternative locations within the eye 10, e.g., within the posterior chamber 11, anterior chamber 12, or in the vitreous humor 13 of the eye 10.

It is noted that relative dimensions in FIG. 1F are not necessarily to scale, but have been rendered for purposes of explanation only in describing the arrangement of the example eye-implantable device 100c within the eye 10. Further, such an implanted device could include multiple elements, located, e.g., in multiple different locations. Such multiple elements could be connected via a cable (e.g., a tether 160) or by some other means. For example, such an implanted device could include a power reception element and controller that is disposed in the posterior capsule 11 and that is operable to receive wireless power from an eye-mountable device or other external system (not shown) and an electrowetting lens that is disposed within the lens capsule 40 could be operated, by the controller, via a tether connecting the controller and the electrowetting lens, using power from the power reception element.

The components of the eye-implantable device 100c may be flexible to permit their being rolled, folded, or otherwise manipulated into a smaller shape to facilitate implantation. This could permit the device 100c to be inserted through a smaller incision through the cornea 20. For example, components of the device 100c could be rolled up, folded in half, folded in thirds, or manipulated in some other way to permit their insertion through an incision 24 that is less than four millimeters long. In some examples, components of the device 100c may be rollable, foldable, or otherwise manipulable such that they can be inserted through an incision 24 that is less than 2 millimeters long. In such examples, the components of eye-implantable device 100 may inserted through the incision 24 in the cornea 20 and/or through some other formed hole or incision (e.g., the hole 25 in the lens capsule) or through some other opening or feature of the eye (e.g., the pupil 26 of the eye 10) to position the device 100c in a specified location of the eye 10. The components could subsequently be unfolded or otherwise manipulated into a flat or otherwise operational state prior to the assembly of an electrowetting lens (e.g., 101) of the eye-implantable device 100c. Such flexibility could improve biocompatibility, speed or otherwise improve the process of implantation, permit detection of forces applied to the device, or could provide some other benefit.

An electrowetting lens (e.g., 101) as described herein may be configured in a variety of ways such that a shape of two or more immiscible fluids (e.g., a polar fluid and a nonpolar fluid) can be controlled by the application of a voltage, current, or other electrical signal to electrodes of the electrowetting lens. In some examples, this could include applying, via the electrodes, an electrical field that changes the effective surface energy, surface tension, interfacial energy, or other surface properties of one or more surfaces within a lens chamber of the electrowetting lens such that a first one of the immiscible fluids retreats or advances across the one or more surfaces. As the first fluid retreats or advances across the one or more surfaces, the overall shape of the first fluid, and of a contact surface between the first fluid and a second fluid that is immiscible with the first fluid, may change. If the first fluid and second fluid have differing refractive indices, light may be refracted when passing through the electrowetting lens and an amount of that refraction (and a corresponding optical power of the electrowetting lens) could be related to the shape of the contact surface. Thus, the overall optical power of the electrowetting lens can be electronically controlled by applying electrical signals to the electrodes of the electrowetting lens to, e.g., control the shape of one or more fluids within the electrowetting lens and/or to control a shape of a contact surface between such fluids of the electrowetting lens.

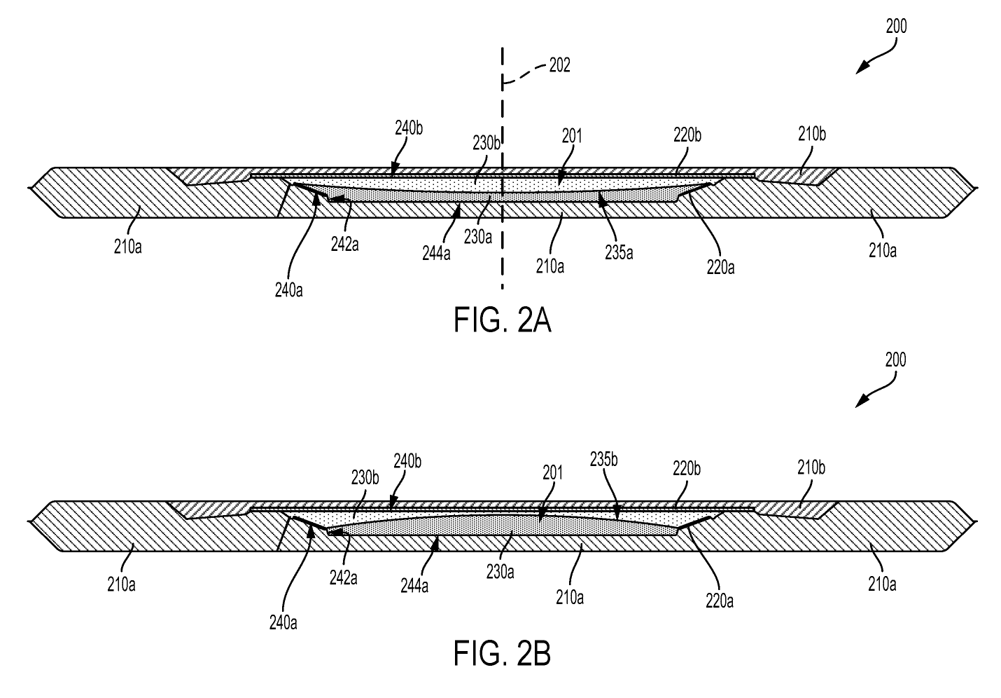

FIG. 2A illustrates a cross-sectional view of an example electrowetting lens 200 during a first period of time. In the example electrowetting lens 200, the lens chamber 201 is radially symmetric about a center line 202. A first electrode 220a is formed along a first internal surface 244a of the lens chamber 201 and takes the form of an inclined ring. A second electrode 220b is formed along a second internal surface 240b of the lens chamber 201. A first fluid 230a is disposed within the lens chamber 201 and, during the first period of time illustrated in FIG. 2A, is in contact with the first internal surface 240a, the first electrode 220a, a third internal surface 242a, and a fourth internal surface 244a of the lens chamber 201. A second fluid 230b is also disposed within the lens chamber 201 and is, during the first period of time, in contact with the second internal surface 240b and the second electrode 220b. During the first period of time, a contact surface between the first fluid 230a and the second fluid 230b has a first shape 235a. The first 230a and second 230b fluids are immiscible (e.g., the first fluid 230a is a nonpolar fluid and the second fluid 230b is a polar fluid) and have differing refractive indices.

The electrowetting lens 200, as illustrated in FIGS. 2A and 2B, includes two immiscible fluids (230a, 230b) which differ with respect to refractive index. Thus, a surface of contact between the first 230a and second 230b fluids (e.g., a concave shape, as shown in FIG. 2A) could provide an optical power (e.g., a diopter, a nonzero focal length) related to the difference in the refractive indices of the fluids 230a, 230b and the shape of the surface of contact.

Introducing fluids 230a, 230b into the lens chamber 201 prior to inserting the electrowetting lens 200 into an eye may cause the fluids to mix, one of the fluids to contact and/or wet a surface that is normally in contact with another of the fluids, or some other unwanted interaction between the fluids and/or surfaces or materials within the lens chamber 201. Such unwanted interactions may result in fouling of surfaces within the lens chamber 201 with one or both of the fluids 230a, 230b, the formation of droplets or foams within the fluids 230a, 230b, a change in the mechanical, chemical or, electrical properties of one or more surfaces within the lens chamber 201 (e.g., a change in the impedance of an electrode surface), a change in the optical properties of the electrowetting lens 200 (e.g., a blurring of image light passed through the lens 200 due to fouling of one or more surfaces within the lens chamber 201), or some other deleterious effects.

To prevent such effects, one or more components of the electrowetting lens 200 could be inserted into an eye before fluid is introduced into the lens chamber 201 and/or before the lens chamber 201 is formed from the one or more components. That is, the electrowetting lens 200 could be manufactured as multiple separate components (e.g., as multiple components that form respective portions of the lens chamber when assembled) without any fluid inside the lens chamber prior to insertion into the eye. Fluid (e.g., the first fluid 230a and/or the second fluid 230b) could subsequently be introduced into the lens chamber 201 and/or onto one or more surfaces that will form part of the lens chamber, once the electrowetting lens 200 is assembled, during in situ assembly of the electrowetting lens 200 from two or more components. For example, a fluid could be introduced after electrowetting lens components have been inserted unrolled, flattened, manipulated to assume a specified shape, manipulated to occupy a specified location within the eye (e.g., a location within the lens capsule of the eye), and/or after they have been assembled to form the completed electrowetting lens 200.

Additionally or alternatively, one or more manipulations of the electrowetting lens 200 could occur after the introduction of a fluid into the electrowetting lens 200. For example, a first fluid 230a may be deposited on surfaces 242a, 244a after the first chamber portion has been manipulated to occupy a specified position within a lens capsule of an eye, but before the insertion of further components and/or the coupling of such further components to the first chamber portion to assemble and/or seal an electrowetting lens chamber. Additional components (e.g., a second chamber portion) may then be interfaced with the first chamber portion containing or otherwise in contact with the first fluid 230a, to form the electrowetting lens 200.

A second fluid 230b could be introduced into the lens chamber 201 via one or more of a variety of methods. In some examples, the electrowetting lens chamber could be formed from two or more components in situ such that the assembly of electrowetting lens 200 causes a second fluid 230b to be trapped in the formed lens chamber 210 in contact with the first fluid 230a. The addition of a second fluid to the electrowetting lens 200 and/or lens chamber 201 thereof could also be achieved by penetrating the lens chamber 201 with a needle and introducing the further fluid 230b into the lens chamber 201 via the needle. Additionally or alternatively, a further fluid could be introduced via a tube or duct that is formed from or otherwise attached to the electrowetting lens 200.

The electrowetting lens 200 could include further elements or features to facilitate the addition, removal, or other interaction with one or more fluids of the electrowetting lens 200 and/or to provide some other benefit(s). For example, the electrowetting lens 200 could include channels, valves, fluidic pathways, microfluidic elements, or other elements or features configured to provide means for fluid to leave the lens chamber 201 or other volumes of the electrowetting lens 200. Such means could be provided to allow fluid (e.g., the second fluid 230b) to exit the lens chamber 201 into the environment of the electrowetting lens 200 (e.g., into a lens capsule of an eye). Such fluid could exit the lens chamber 201 as a result of the introduction of fluid into the lens chamber 201, e.g., via a septum, a tube, or some other means. Other methods may also be used to introduce one or more fluids into the electrowetting lens 200.

The introduced fluid could be a fluid not previously present in the lens chamber 201 (e.g., an oil or other component of the first fluid 230a that was omitted from the electrowetting lens 200 prior to implantation and/or assembly to prevent fouling of surfaces of the lens chamber 201 during implantation of the electrowetting lens 200 or to provide some other benefit). Additionally or alternatively, an introduced fluid could be of the same type as a fluid already present in the electrowetting lens 201, e.g., to rinse out the lens chamber 201, to remove gases (e.g., gases dissolved in the second fluid 230b that could form bubbles within the lens chamber 201) from the lens chamber 201, to control a volume or internal pressure of the lens chamber 201, or to provide some other benefit.

As the first 230a and second 230b fluids differ with respect to refractive index, light that passes through the contact surface (e.g., light that is passing through the electrowetting lens 200 along the center line 202) may be refracted. A degree or amount of the refraction, and a related optical power of the electrowetting lens 200, may be related to the shape of the contact surface between the first fluid 230a and the second fluid 230b.

The refractive indices of the two fluids 230a, 230b may differ by a specified amount. The optical power of the electrowetting lens 200 (e.g., the controllable range of optical powers of the electrowetting lens 200) may be related to the magnitude of the difference between the refractive indices. The refractive indices of the two fluids 230a, 230b could differ by more than 0.1. The difference between the refractive indices could be controlled by controlling and/or modifying the refractive index of one or both of the fluids 230a, 230b.

The refractive index of an aqueous fluid (e.g., the second fluid 230b) may be approximately equal to 1.33, the refractive index of water. Alternatively, butanediol or some other substance(s) could be added to such an aqueous solution such that the refractive index of the aqueous solution differs from 1.33. In examples where a substance is added to an aqueous (or other) fluid of the electrowetting lens 200, the lens chamber of the electrowetting lens 200 may include a seal or coating (e.g., could be hermetically sealed) to prevent such a substance from exiting the electrowetting lens 200 and entering the aqueous humor of an eye.

Properties of a nonpolar fluid (e.g., the first fluid 230a) could additionally or alternatively be specified to control the refractive index of the nonpolar fluid. This could include adding substances to the nonpolar fluid. For example, a phenylated silicone oil (e.g., polyphenylmethylsiloxane) could be added to a silicone oil (e.g., to polydimethylsiloxane) to increase its refractive index and/or density. Additionally or alternatively, a ratio of components of a nonpolar fluid could be specified to control the refractive index of the nonpolar fluid. For example, a ratio between a first linear alkane (e.g., hexadecane) and a second linear alkane (e.g., nonadecane) could be specified to control the refractive index of the nonpolar fluid. Yet further, a polymer length, a polydispersity, a degree of branching, or some other properties of a nonpolar fluid could be specified to control the refractive index of the nonpolar fluid and/or to control some other property (e.g., melting point, viscosity, surface energy, density) of the nonpolar fluid.

The shape of the contact surface can be controlled by applying an electrical signal to the electrodes 220a, 220b, e.g., by applying an electrical voltage to the electrodes 220a, 220b. The voltage applied to the electrodes 220a, 220b may be related to the steady-state (e.g., following any transient changes in the electrowetting lens resulting from changes in the applied voltage) optical power of the electrowetting lens 200 and/or the shape of the contact surface between the fluids 230a, 230b. The specific relationship could be based on an effect on the surface energy of the first internal surface 240a relative to each of the fluids 230a, 230b, to an effective capacitance between the first electrode 220a and the second electrode 220a via a conductive second fluid 230b (e.g., via a second fluid 230b that includes a conductive, aqueous solution and that is in conductive and/or capacitive electrical contact with the second electrode 220b), or to some other factors.

The first electrode 220a and second electrode 220b could include conductive materials (e.g., aluminum, gold, copper, or other materials) disposed on respective internal surfaces of the lens chamber 201 (e.g., on surfaces of the first element 210a and second element 210b, respectively). One or both of the electrodes could further include a dielectric layer disposed between such a conductive material and the inside of the lens chamber 201. For example, the first electrode 220a could include such a dielectric layer. Such a dielectric layer could be provided to prevent large, direct currents from passing from the first electrode 220a into one or both of the first 230a or second 230b fluids, to provide a capacitive electrical coupling between the first electrode 220a and such fluids, to limit an amount of charge that can be transmitting into such fluids via the first electrode 220a, or to provide some other benefits.

Such a dielectric layer could be a separate material (e.g., parylene) deposited on the conductive material (e.g., via CVD, spin coating, or some other process). Additionally or alternatively, the dielectric layer of the first electrode 220a could be formed from the conductive material of the electrode, e.g., the dielectric layer could be a nonconductive layer of aluminum oxide formed by oxidation of an underlying aluminum metal of the first electrode 220a. Such a dielectric layer could be formed via anodization or other electrically-driven reactions at the surface of the electrode. Additionally or alternatively, such a dielectric layer could be formed by redox reactions between the fluids in the lens chamber 201 and the material of the electrode.

In some examples, the formation and/or maintenance of such a dielectric layer could be negatively impacted by the presence of certain ions within the lens chamber 201 (e.g., dissolved in one or both of the fluids 230a, 230b). For example, the presence of chloride ions could act to pit or otherwise damage a dielectric layer of aluminum oxide that has formed on the surface of an aluminum electrode. In such examples, a barrier could be formed from a chloride-impermeable material to prevent chloride ions present in the aqueous humor (or in some other environment to which the lens 200 is exposed) from entering the lens chamber 201 or from entering some other material or volume of the lens 200. Such a material could include a polymeric material, a metal foil or deposited metal layer, or some other material(s). Such materials could be substantially transparent to visible light.

The voltage between the electrodes 220a, 220b could be controlled in order to control the optical power of the electrowetting lens 200 by controlling the shape of the contact surface between the fluids 230a, 230b. FIG. 2B illustrates the electrowetting lens 200 during a second period of time during which a voltage is being applied to the electrodes 220a, 220b such that the contact surface between the first fluid 230a and the second fluid 230b has a second shape 235b. As a result, the optical power of the electrowetting lens 200 during the second period of time is different than the optical power of the electrowetting lens 200 during the first period of time.

The particular shape of the contact surface and/or of the geometry of the fluids 230a, 230b could be related to the applied voltage and to a variety of other factors. Such factors could include the interfacial energy between the fluids 230a, 230b, the interfacial energy between the fluids 230a, 230b and the internal surfaces 240a, 242a, 244a, 240b, the geometry of the internal surfaces 240a, 242a, 244a, 240b, a geometry of the electrodes 220a, 220b, and/or a geometry of a dielectric layer of the first electrode 220a. One or more of these factors could be specified in order to affect the shape of the contact surface between the fluids 230a, 230b, to affect the geometry and/or location of the fluids 230a, 230b within the lens chamber 201, to affect the relationship between an applied voltage and the optical power of the electrowetting lens 200, or to affect some other property of interest of the electrowetting lens 200.

This could include adding surfactants, polar and/or ionic substances, nonpolar substances, to the fluid(s) or otherwise specifying a composition of the first 230a and/or second 230b fluids to control an interfacial energy between the fluids 230a, 230b and/or to control an interfacial energy between the fluids and the internal surfaces 240a, 242a, 244a, 240b of the lens chamber. Additionally or alternatively, the composition of the material composing the internal surfaces 240a, 242a, 244a, 240b could be specified to control the interfacial energy between the internal surfaces and the fluids.

This could include selecting the bulk materials of the first 210a and second 210b elements and/or providing one or more coatings or surface treatments to the internal surfaces of the lens chamber 201. For example, the first fluid 230a could be an oil or other nonpolar fluid and one or more of the first 240a, third 242a, or fourth 244a internal surfaces could be superhydrophobic or otherwise hydrophobic. Further, the second fluid 230b could be a polar fluid (e.g., could include a saline solution or other aqueous solution) and the second 240b internal surface could be underwater oleophobic, underwater superoleophobic, hydrophilic and/or superhydrophilic (e.g., by including a surface coating, by including surface features or textures, by having been exposed to an oxidization process, or by some other means).

The distribution of such coatings or materials on the internal surfaces of the lens chamber 201 and/or the geometry of such surfaces could be specified to center the first fluid 230a along the center line 202 or along some other specified axis of the electrowetting lens 200. This could include applying different coating or other material to internal surfaces according to distance from the center line 202. Additionally or alternatively, a thickness or other property of a dielectric of the first electrode 220a could vary according to distance from the center line 202 such that, when a voltage is applied between the electrodes 220a, 220b, electrical and/or interfacial forces applied to the first 230a and/or second 230b fluids tend to center the first fluid 230a along the center line 202 and/or to conform a boundary between the fluids 230a, 230b on the first internal surface 240a to a circle centered on the center line 202.

The lens chamber 201 could be permeable to water or other substances (e.g., ions) in aqueous humor of an eye. This could include the lens chamber 201 being composed at least partially of a polymeric material that is permeable to water (or other substances) in the aqueous humor. In examples wherein the lens chamber is permeable to a substance that is present in the aqueous humor, one or both of the fluids 230a, 230b could include a concentration of the substance corresponding to the concentration of the substance in the aqueous humor, e.g., to prevent a net flow of the substance from the aqueous humor into the lens chamber 201 or vice versa.

Additionally or alternatively, the lens chamber could be made impermeable to such substances in the aqueous humor and/or to substances in one or both of the fluids 230a, 230b. For example, one of the fluids could be a conductive fluid that includes butanediol, and the lens chamber could be made impermeable to butanediol and/or could be hermetically sealed. This could include constructing the lens chamber from materials that are impermeable to the substances. Additionally or alternatively, a barrier layer or coating could be formed from such impermeable materials to prevent the substances from entering the lens chamber 201 or some other element or structure of the electrowetting lens 200. For example, a barrier could be formed from a chloride-impermeable material to prevent chloride ions present in the aqueous humor from entering the lens chamber 201 or from entering some other material or volume of the lens 200. Such a material could include a polymeric material, a metal foil or deposited metal layer, or some other material(s). Such materials could be substantially transparent to visible light.

### III. EXAMPLE IN SITU ASSEMBLY OF AN ELECTROWETTING LENS

To facilitate implantation of an eye-implantable device, it can be beneficial for the device to be composed of multiple components that may be assembled in situ within the eye. Components of such a device can be inserted individually into the eye and positioned or otherwise manipulated prior to being assembled to form the eye-implantable device. For example, a first component, which includes a first lens chamber portion, can be positioned within the lens chamber of the eye. Subsequently, fluids, electronic components, and/or further components (e.g., components including respective further lens chamber portions) may be inserted or otherwise introduced into the eye and assembled together to form an eye-implantable device as described herein.

In situ assembly could allow insertion of components of the device into the eye through an incision that is smaller than the assembled device and/or than a characteristic size of the assembled device when rolled, folded, or otherwise configured to facilitate such insertion. In situ assembly may also reduce a chance of failure of the device or otherwise improve operation of the device following implantation. For example, components of the electrowetting lens could be inserted into the eye without one or more immiscible fluids of the electrowetting lens. These fluids could be added later, preventing the fluids from mixing or otherwise dispersing into each other during insertion, from contacting and wetting particular surfaces within the assembled electrowetting lens, or from producing other deleterious effects that could affect the optical properties or functioning of the assembled electrowetting lens. In situ assembly may reduce the mechanical and/or chemical seal requirements of the device relative to a device that is not assembled in situ and that is inserted as a pre-assembled fluid-filled electrowetting lens. In situ assembly may also allow for modular device design such that individual device components may be specified and/or selected according to a particular user (e.g., based on a specified optical power, size, fluid volume, coloration, or other consideration of the particular user).

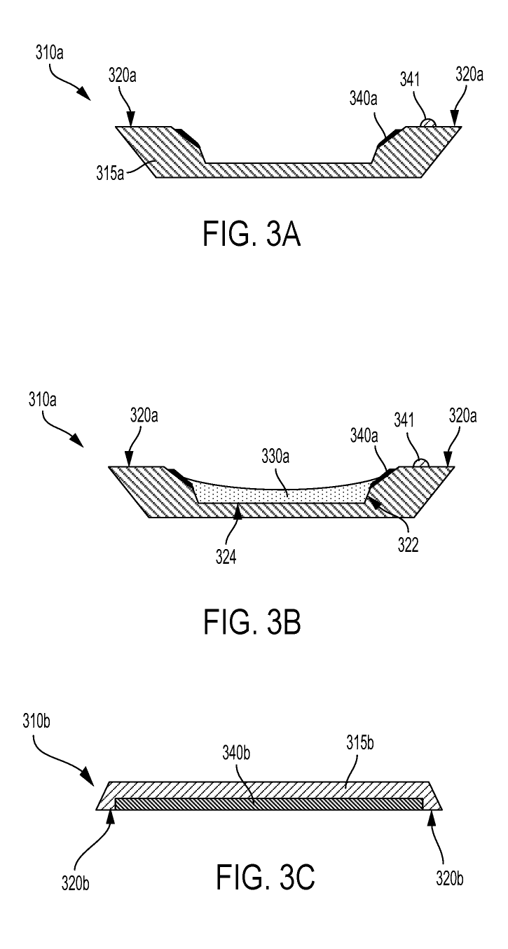

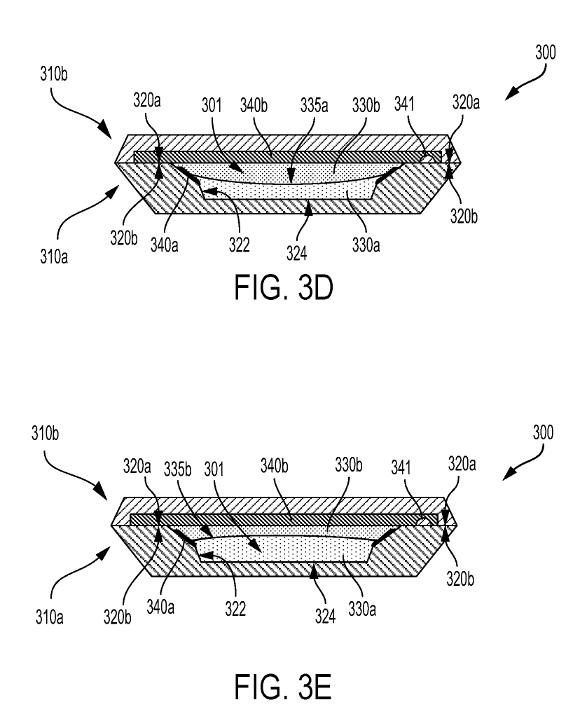

FIG. 3A is a cross-sectional view of a first lens chamber portion 310a of an eye-implantable device. The lens chamber portion includes a transparent polymer 315a, an inclined ring-shaped electrode 340a, an electrical contact 341, and a first mating surface 320a. In some embodiments, the first chamber portion 310a may also include a second electrode, a controller electronically coupled to the first and/or second electrodes, a tether, sensors, antennae, other electronic components, or additional components which may facilitate the functioning of an electrowetting lens formed from the first lens chamber portion 310a.

FIG. 3B shows the first chamber portion 310a of FIG. 3A with a first fluid 330a deposited on a surface of the first chamber portion such that it is in contact with chamber surfaces 322, 324 and electrode 340a. In some embodiments, this fluid is an oil or other nonpolar fluid. Such a fluid 330a could be introduced after the first chamber portion 310a has been inserted, unrolled, flattened, manipulated to assume a specified shape and/or manipulated to occupy a specified location within the eye (e.g., a location within the lens capsule of the eye).

Alternatively, the first fluid 330a may be deposited onto the first chamber portion 310a prior to insertion of the first chamber portion 310a into an eye such that the insertion of the first chamber portion 310a also inserts the fluid 330a into the eye. In some examples, this could include disposing the first fluid 330a or some other precursor substance on the first chamber portion 310a in a frozen or otherwise solid or semi-solid state (e.g., a gel, a sol-gel). Such a substance could have a melting point or other characteristic temperature that is less than body temperature (e.g., less than 37° C.) such that the disposed fluid melts, exhibits a reduction in viscosity, or otherwise becomes fluid after the first chamber portion 310a is inserted into the eye. Such a first chamber portion 310a could be maintained at a temperature below the melting point of the first fluid 330a prior to and/or during insertion of the first chamber portion 310a into an eye (e.g., by being disposed within a refrigerated container, by inserting the first chamber portion 310a using a cooled instrument, by irrigating the eye with chilled saline or other fluid or otherwise reducing the temperature of the eye). Additionally or alternatively, the melting point or other characteristic temperature could be greater than room temperature (e.g., greater than 20° C.) such that maintaining the first chamber portion 310a at a temperature below the melting point of the first fluid 330a could include exposing the first chamber portion 310a to ambient conditions in a room.

FIG. 3C shows a cross-sectional view of a second chamber portion 310b of an eye-implantable device. The second chamber portion 310b includes a transparent polymer 315b, a second electrode 340b and a second mating surface 320b shaped to mate with the first mating surface 320a of the first chamber portion 310a. Note that the illustrated second chamber portion 310b is intended as a non-limiting example of a further chamber portion that could be mounted to the first chamber portion 310a. The second chamber portion 310b may additionally include a controller, a tether, sensors, antennae, other electronic components, or additional components (not shown) which may facilitate the functioning of an electrowetting lens 300 formed from the first lens chamber portion 310a and second chamber portion 310b.

FIG. 3D is a cross-sectional view of an assembled electrowetting lens 300 formed when the first chamber portion 310a is mated with a second chamber portion 310b. As illustrated in FIG. 3D, mating of the first 310a and second 310b chamber portions forms an electrowetting lens 300 comprising a lens chamber 301, chamber surfaces 322, 342, a first and second fluid 330a, 330b, and at least a first and second electrode 340a, 340b.

Mating of the first 310a and second 310b chamber portions could result in electronic coupling of the first 340a and second 340b electrodes and/or other electronic components (e.g., controllers, sensors, antennae) of the first and/or second chamber portions. Electronic coupling could facilitate the application of voltage between the first 340a and second 340b electrodes (e.g, to detect an analyte of interest), communication between electronic components of the eye-implantable device, the storage or retrieval of data, or some other function. In order to electronically couple components of the first 310a and second 310b chamber portions, one or more of the portions could include an electrical contact 341. For example, the first 310a and/or second 310b chamber portions could include one or more penetrating needles, pins, or other means to facilitate an electrical connection between such components and the first and/or second electrode 340a, 340b.

The first chamber portion 310a and/or the second chamber portion 310b could be composed of a flexible polymer (e.g., the transparent polymer 315a could be a flexible polymer) capable of being folded, rolled, bent, or otherwise manipulated. Such manipulation could permit the first 310a and/or second 310b chamber portions to be inserted via an incision that is smaller than a diameter or other characteristic dimension of the first 310a and/or second 310b chamber portions. For example, the first 310a and/or second 310b chamber portions could be rolled, folded, or otherwise manipulated such that they can each be inserted through an incision that is less than 4 millimeters long, or through an incision that is less than 2 millimeters long. Once inserted into an eye (e.g., via an incision), the first 310a and/or second 310b chamber portions may be positioned, flattened, or otherwise manipulated to facilitate the assembly or operation of the electrowetting lens 310.

To form the electrowetting lens 300, the first mating surface 320a of the first chamber portion 310a is mated with the second mating surface 320b of the second chamber portion 310b. As shown in FIG. 3D, bringing the first mating surface 320a into contact with the second mating surface 320b results in the formation and enclosure of a lens chamber 301 of the electrowetting lens 300. Such a lens chamber is formed such that respective chamber surfaces 322, 324 form internal surfaces of the formed lens chamber 301, and the first 330a and second 330b electrodes are disposed on respective internal surfaces of the lens chamber.

The mating surfaces 320a, 320b comprise respective surfaces of the first 310a and second 310b chamber portions which are shaped to mate together to form the electrowetting lens 310. For example, the first mating surface 320a may be a flat circular surface along the circumference of the first chamber portion, which may be in contact with the second mating surface 320b on the second chamber portion 310b when the first 310a and second 310b chamber portions are mated together to form the electrowetting lens 310. In some examples, the mating surfaces 320a, 320b may only partially encircle the lens chamber 301, or may comprise two or more non-contiguous surfaces. Such a mating surface can further include one or more clamping features, a pressure-sensitive adhesive, an amount of meltable adhesive, or some other means for coupling the first and second chamber portions together and/or for preventing the flow of fluid into or out of the lens chamber 301. The first and second mating surfaces 320a, 320b may also include alignment features, such as pins, ridges, grooves, or other structures, that could facilitate the assembly, alignment, and/or sealing of the electrowetting lens 310 and/or of the lens chamber 301 thereof.

When the first chamber portion 310a is mated with the second chamber portion 310b, the movement of fluid between the lens chamber 301 and the outside environment may be inhibited. In some embodiments, the lens chamber 301 formed from the mating of the first 310a and second 310b chamber portions may be sealed hermetically such that no fluid can enter and/or exit the lens chamber. In other embodiments, the mating of the first chamber portion 310a with the second chamber portion 310b may allow for some movement of fluid (e.g., the first fluid 310a, the second fluid 310b, or fluid from the environment) into or out of the lens chamber.

As shown in FIG. 3D, the assembled electrowetting lens 310 includes a second fluid 330b contained within the lens chamber 301. The first 330a and second 330b fluids could be immiscible and differ with respect to refractive index (e.g., saline or another aqueous fluid and oil or another non-polar fluid). Thus, a surface of contact 335a between the first 330a and second 330b fluids could provide an optical power (e.g., a diopter, a nonzero focal length) related to the difference in the refractive indices of the fluids 330a, 330b and the shape of the first contact surface 335a.

Such a second fluid 330b may be introduced into and/or otherwise disposed within the lens chamber 301 of the electrowetting lens 310 in a variety of ways. For example, the second fluid 330b may be introduced into an environment of the first chamber portion 310a (e.g., into a lens capsule, a posterior chamber, or some other environment containing the first chamber portion 310a) prior to insertion and/or mating of the second chamber portion 310b to the first chamber portion 310a. In such an example, coupling the first chamber portion 310a and the second chamber portion 310b together to form the electrowetting lens 300 causes the second fluid 330b (e.g., an amount of a saline fluid introduced into the environment of the first chamber portion 310a), to be contained within the formed lens chamber 310 in contact with the first fluid 330a and the second electrode 340b. Such a fluid could be provided to rinse debris from the environment of the first chamber portion 310a (e.g., amounts of a viscoelastic gel provided to maintain a volume or geometry of a lens capsule following removal of a crystalline lens, fragments of a phacoemulsified or otherwise disintegrated crystalline lens, cellular debris), to purge dissolved gases from fluid in the environment of the first chamber portion 310a, to de-bubble one or more surfaces of the first 310a and/or second 310b chamber portions, or to provide some other benefit additional to providing the second fluid 330b that is disposed within the lens chamber 301 of the formed electrowetting lens 300.

Additionally or alternatively, the second fluid 330b and/or the first fluid 330a may be introduced into the lens chamber 301 of the electrowetting lens 300 after the lens chamber 301 has been formed. The addition of one or more fluids (e.g., 330a, 330b) to the electrowetting lens 300 and/or the lens chamber 301 thereof could be achieved by penetrating the lens chamber 301 or a septum or other element of the electrowetting lens 300 in fluid communication with the lens chamber 301 with a needle. The fluid(s) may then be introduced into the lens chamber 301 via the needle. In another example, the fluid(s) could be introduced via a tube or duct that is formed from or otherwise attached to the electrowetting lens 300. Means for adding fluid(s) to the electrowetting lens 300 could also be used to remove an amount of fluid from the electrowetting lens 300 (e.g., from the lens chamber 301). This could be performed to control a volume of the lens chamber 301, to allow rinsing, de-bubbling, or de-gassing of fluid(s) within the lens chamber 301, to allow replacement of a fluid disposed within the lens chamber 301 with an alternative fluid (e.g., to remove a chloride-containing saline solution used to irrigate the eye with a chloride-free aqueous saline solution), or to provide some other benefits.

As shown in FIG. 3D, the second chamber portion 310b comprises a second electrode 340b which is in electrical contact with the electrical contact 341 when the first chamber portion 310a is mated with the second chamber portion 310b. Such electrical contact could facilitate application of voltages, currents, or other electrical signals to the second electrode 340b by controllers or other electronics disposed within the first chamber portion 310a or otherwise electrically coupled to the electrical contact 341. Such a controller or other electronics could also be electrically coupled to the first electrode 340a to facilitate electronic control of the overall optical power of the electrowetting lens 300 by controlling the geometry of the contact surface between the first fluid 330a and the second fluid 330b.

This is illustrated in FIG. 3E, where a voltage and/or current has been applied to the electrodes 340a, 340b such that first 330a and second 330b fluids are in contact along a second contact surface 335b. The applied voltage and/or current can be specified to control a shape of the contact surface between the two fluids 330a, 330b in order to control an overall optical power of the electrowetting lens 300. As shown in FIGS. 3D and 3E, this can include changing the shape of the contact surface between the first 330a and second 330b fluids from a concave shape (e.g., 335a) to a convex shape (e.g., 335b).

In situ assembly of an eye-implantable device as described herein may also include washing, irrigation and/or debubbling of a region within an eye to remove debris, clear excess viscoelastic gels, improve the optical clarity of the assembled device, or to provide some other benefit. Verification testing can also be performed to determine whether the assembled device is functional, to adjust individual components, or to calibrate the device (e.g., to determine the optical power as a function of voltage applied). This could include applying a voltage across the electrodes and assessing the optical power of the assembled electrowetting lens as a function of the applied voltage (e.g., using Shack-Hartmann aberrometry, a Tscherning system, ray tracing, sciascopy, or some other method of detecting the optical properties of such a device while located within an eye). Other methods of in situ assembly, manipulation, and/or testing of eye-implantable device components as described herein are contemplated.

An eye-implantable device as described herein could be assembled from two or more components; such components could include respective portions of a lens chamber of an electrowetting lens, two or more electrodes, and at least one fluid disposed on a surface within a lens chamber. In some examples, two or more chamber portions comprising respective mating surfaces assemble together to form respective portions of the electrowetting lens of the eye-implantable device. For example, a first 110a and second 110b chamber portion comprising electronics, lenses, fluids, and/or other components may be mated within the eye to form the electrowetting lens 101 of the eye-implantable device 100a. Such chamber portions could be configured in a variety of ways such that they may be mated along respective mating surfaces to assemble the eye-mountable device. Such eye-mountable devices could include features or elements configured to securely couple such components together, to provide a seal (e.g., to inhibit the flow of fluids into or out of one or more fluid-filled chambers of the device), to secure the device in a specified location within an eye, to transmit forces from the eye to the device (e.g., accommodation forces exerted on a lens capsule that could be detected by the device and used to operate an electrowetting lens), or to provide some other benefit. For example, one or more of the components forming an eye-mountable device could include snap-fit features and/or materials having specified moduli of elasticity to facilitate mechanical coupling of the components to each other. Additionally or alternatively, adhesives, clamps, or other elements could be added to and/or form part of the components to facilitate mechanical coupling of the components to each other, to seal a lens chamber, or to provide some other benefit. An eye-implantable device could include additional elements or features according to an application.

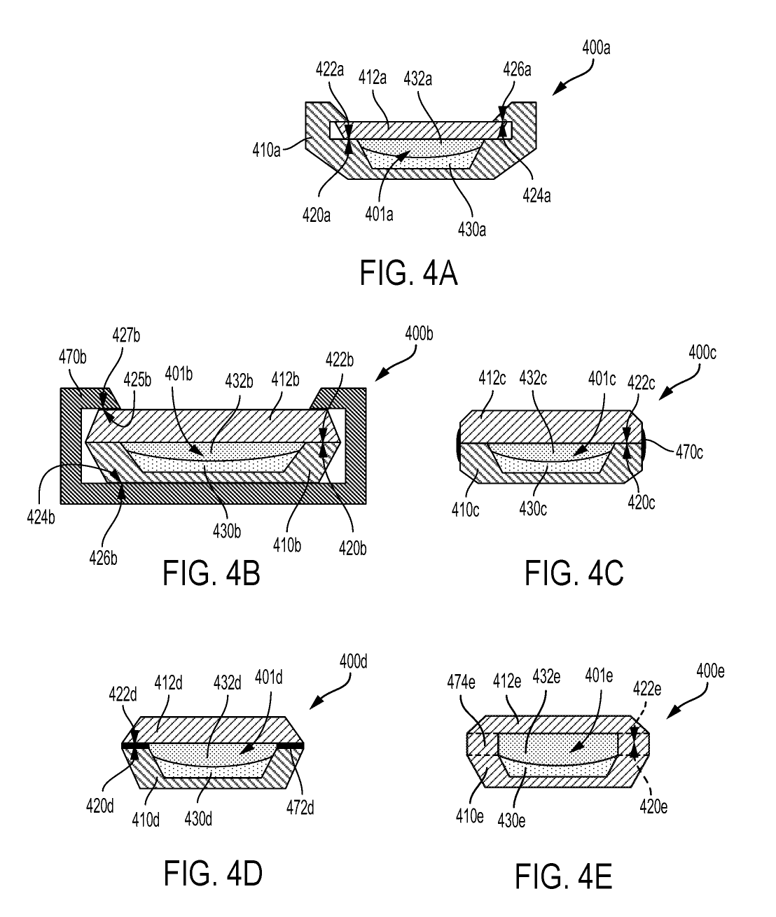

Components of an eye-implantable device as described herein could include clamping features to facilitate securing the components together to form the eye-implantable device. Such clamping features could include pins, snaps, buttons, ridges, grooves, teeth, threads, or other formed features or elements. FIG. 4A shows, in cross-section, an eye-implantable device 400a assembled from a first chamber portion 410a and a second chamber portion 412a. The first 410a and second 412a chamber portions are mated along respective first 420a and second 422a mating surfaces to form a lens chamber 401a of an electrowetting lens of the eye-implantable device 400a. The lens chamber 401a contains first 430a and second 432a immiscible fluids. The first chamber portion 410a includes a first clamping feature 426a (e.g., an overhanging ridge, edge, lip, tooth, clip, or snap) and the second chamber portion 412a includes a second clamping feature 424a (e.g., an edge, prong, ridge, or other feature that interacts with the first clamping feature 426a). The first chamber portion 410a and the second chamber portion 412a are mechanically coupled via the first clamping feature 426a and the second clamping feature 424a.

Clamping features (e.g., 424a, 426a) of an eye-implantable device may exert residual forces on each other in order to mechanically couple the first 410a and second 412a chamber portions together. Such clamping features may take the form of one or more clamping arms, ridges, tabs, ridges, edges, tapered holes, tapered pins, pins, teeth, threads, grooves, or other structures of the first and/or second chamber portions 410a, 412a. Such clamping feature(s) may comprise the same material as the first and/or second chamber portions 410a, 412a of which they are part, or may comprise a different material. Similarly, the first and/or second chamber portions 410a, 412a may comprise the same material or may comprise different materials. For example, one or more materials of the first and/or second chamber portions 410a, 412a and/or clamping features thereof (e.g., 424a, 426a) could be specified to have properties that facilitate mechanical coupling of the chamber portions (e.g., via a snap fit). This could include the first and/or second chamber portions 410a, 412a and/or clamping features 424a, 426a thereof being comprised of respective different materials having respective different moduli of elasticity such that, when the first mating surface 420a is mated with the second mating surface 420b, a force is exerted between the first clamping feature and the second clamping feature.

In some examples, components of such an eye-implantable device may further include a clamp, which could apply a force to mechanically couple the first 410a and second 410b chamber portions together. FIG. 4B illustrates a cross-sectional view of an eye-implantable device 400b assembled from a first chamber portion 410b, a second chamber portion 412b, and a clamp 470b. The first 410b and second 412b chamber portions are mated along respective first 420b and second 422b mating surfaces to form a lens chamber 401b of an electrowetting lens of the eye-implantable device 400b. The lens chamber 401b contains first 430b and second 432b immiscible fluids. The first chamber portion 410b and the second chamber portion 412b comprise respective first 424b and second 425b clamping surfaces. Such clamping surfaces 424b, 425b may be flat circular or ring-shaped exterior surfaces along the circumference of the eye-implantable device 400b (i.e., around the circumference of the first 410b and second 412b chamber portions). In some embodiments, the clamping surfaces 424b, 425b may comprise only a portion of the circumference of the first 410a and second chamber portions, or one or more discontinuous regions,

The clamp 470b includes a third clamping surface 426b that contacts the first clamping surface 424b. The clamp 470b also includes a fourth clamping surface 427b that contacts the second clamping surface 425b. In such an example, the clamp 470b could apply forces to the first clamping surface 424b and the second clamping surface 425b when the first mating surface 420a is mated with the second mating surface 422b, the first clamping surface 424b is in contact with the third clamping surface 426b, and the second clamping surface 425b is in contact with the fourth clamping surface 427b. Such forces, applied by the clamp 470b to the first 410b and second 412b chamber portions, could act to mechanically couple the first 410a and second 410b chamber portions together.

A clamp (e.g., 470b) of such an eye-implantable device may have a structure that encircles substantially all of the first and/or second mating surfaces, such that a force is applied around the circumference of one or both chamber portions 410a, 410b of the electrowetting lens 410. The clamp 470b could also comprise a smaller structure, which exerts force on a smaller region or regions of the first 424b, second 425b, third 426b, or fourth 427b clamping surfaces when the first mating surface 420b of the first chamber portion 410b is mated with the second mating surface 420b of the second chamber portion 410b. The clamp 412 may be one component, or a series of components which exerts a force at one or more points along the circumference of the electrowetting lens. For example, a one or more clamps 470b could include a hinge (e.g., a catch), and could mechanically couple the first 410b and second 412b chamber portions by exerting a force on the first 424b and third 425b clamping surfaces via the hinge. A further clamp could exert a further force on a region of the clamp(s), components of the eye-implantable device, or another region to mechanically couple the eye-implantable device 400b (e.g., to prevent the hinge from opening). The first 410a or second 410b chamber portion may also contain pilot holes, ridges or other apertures that facilitate the placement of the clamp 470b.

Such a clamp may be inserted into an eye during an implantation process and subsequently used to mechanically couple the first 410b and second 412b chamber portions together. In some examples, the clamp could be inserted into the eye before any other components of the eye-implantable device 400b, so that it may act as a base on which to assemble the first chamber portion 410b, the second chamber portion 412b, and/or additional components of the eye-implantable device 400b. In other embodiments, the clamp 470b may be inserted after at least one of the components of the eye-implantable device 400b (e.g., the first or second chamber portions 410b, 412b) has been inserted, positioned, manipulated, or assembled in situ. Such a clamp 470b could be later removed from the first 410b and second 412b chamber portions (e.g., by cutting, crimping, laser cutting, dissolving, or some other means) or could remain as part of the eye-implantable device 400b following implantation and assembly. Such a clamp may be composed of the same material as a component of the eye-implantable device 400b (e.g., as the first or second chamber portions 410b, 412b) or a different material with properties that facilitate the insertion, attachment, assembly, and/or removal of the clamp 470b.

In some examples, components of such an eye-implantable device may include a sealant, which could be applied to components of an eye-implantable device to mechanically couple the components together, to inhibit fluid flow into or out of one or more volumes of the eye-implantable device (e.g., a lens chamber of an electrowetting lens), or to provide some other benefit. FIG. 4C illustrates a cross-section view of an eye-implantable device 400c assembled from a first 410c and second 412c chamber portion, mated to form a lens chamber 401c using a sealant 470c. The first 410c and second 412c chamber portions are mated along respective first 420c and second 422c mating surfaces to form a lens chamber 401c of the eye-implantable device 400c. The lens chamber 401c contains a first 430c and second 432c immiscible fluids. The first 410c and second 412c chamber portions are mechanically coupled via a sealant 470c.

When applied to one or more of the first or second chamber portions, such a sealant 470c could inhibit fluid flow between the lens chamber 401c and an external environment of the eye-implantable device 400c, could mechanically couple the first 410c and second 412c chamber portions together, or could provide some other benefit. The sealant could be applied to one or more surfaces of the first 410c and/or second 412c chamber portions when the first mating surface 420c of the first chamber portion 410c is mated with the second mating surface 422c of the second chamber portion 412c. For example, a sealant 470c could be applied around the circumference of the formed eye-implantable device 400c, such that the sealant 470c is in contact with both the first 410c and second 412c chamber portions. The sealant 470c may include a biocompatible, saline-compatible material, for example a silicone (e.g., polydimethylsiloxane, PDMA), an epoxy, a urethane, or another polymer sealant. Such a sealant 470c could be cured. Such curing could include applying light (e.g., ultraviolet light), heat, a reagent, or some other energy or substance to facilitate curing of the sealant. Additionally or alternatively, the sealant 470c could be self-curing, and curing the sealant could include allowing the sealant to cure for a specified period of time.

In some examples, one or more components of an eye-implantable device could include a layer of pressure-sensitive adhesive that could facilitate assembly of the eye-implantable device, mechanical coupling of components of the eye-implantable device, inhibition of fluid flow into or out of one or more volumes of the eye-implantable device (e.g., a lens chamber of an electrowetting lens), or could provide some other benefit. FIG. 4D illustrates a cross-sectional view of an eye-implantable device 400d assembled from a first 410d and second 412d chamber portion mated to form a lens chamber 401d using a pressure-sensitive adhesive 472d. The first 410d and second 412d chamber portions are mated along respective first 420d and second 422d mating surfaces to form a lens chamber 401d of the eye-implantable device 400d. The lens chamber 401d contains a first 430d and second 432d immiscible fluids. The first 410d and second 412d chamber portions are mechanically coupled via a pressure-sensitive adhesive 470d.

Such a pressure-sensitive adhesive 472d may be disposed on at least one of the first mating surface 420d of the first chamber portion 410d or the second mating surface 422d of the second chamber portion 412d. For example, the pressure-sensitive adhesive 472d could be disposed on the first mating surface 420d of the first chamber portion 410d, such that it is disposed between the first 420d and the second 422d mating surfaces of the first 410d and second 412d chamber portions when they are mated to form the eye-implantable device 400d. Additionally or alternatively, a pressure-sensitive adhesive 472d may be applied to the second mating surface 422d. The pressure-sensitive adhesive 472d may further comprise a release liner, which could prevent nonspecific adhesion of the pressure-sensitive adhesive to a different region of the eye-implantable device 400d, to a region of the eye, or to some other location. Such a release liner may be removed prior to insertion of the first 410d and/or second 412d chamber portions into the eye. Alternatively, the release liner may be removed after at least one component of the eye-implantable device 400d (e.g., the first 410d and/or second 412d chamber portion) has been inserted into the eye, but before the mating of the first chamber portion 410d and the second chamber portion 412d and/or the assembly of the eye-implantable device 400d.

In some examples, such an adhesive material could include a self-healing polymer. Such a self-healing polymer could be disposed on the first mating surface 420d of the first chamber portion 410d and/or the second mating surface 422d of the second chamber portion 412d such that the amounts of self-healing polymer of each of the chamber portions 410d, 412d join together when the first 410d and second 412d chamber portions are mated to form the eye-implantable device 400d. This could include the bulk material of the first 410d and/or second 412d chamber portions comprising such a self-healing polymer to promote adhesion of the chamber portions, prevent damage to the first and/or second chamber portions, to improve the lifespan of the eye-implantable device 400d, or to provide some other benefit.

An adhesive material of such a component of an eye-implantable device could include a meltable adhesive. Heat could be applied to such a meltable adhesive (e.g., by illumination by a laser) to melt the meltable adhesive, while in an eye, such that two or more components of the eye-implantable device are mechanically coupled together, a volume of the eye-implantable device is sealed, or such that two or more components of the eye-implantable device are otherwise adhered together. An amount of such a meltable adhesive could be disposed on a material of a component of the eye-implantable device (e.g. on a mating surface of such a component). Alternatively, such a component could be composed of such a meltable adhesive material and a specified region of the component could be melted (e.g., by applying heat specifically to the specified region) in order to adhere the component to one or more other components of the eye-implantable device.

Additionally or alternatively, one or more components of such an eye-implantable device could comprise a meltable adhesive material 474e, and coupling of the device components could include a heat-sealing approach. FIG. 4E illustrates a cross-sectional view of an eye-implantable device 400e assembled from a first 410e and second 412e chamber portion mated to form an electrowetting lens via a heat-sealing approach. The first 410e and second 412e chamber portions are mated along respective first 420e and second 422e mating surfaces to form a lens chamber 401e of the eye-implantable device 400e. The lens chamber 401e contains a first 430e and second 432e immiscible fluids. The first 410e and second 412e chamber portions are mechanically coupled via melting (i.e., heat-sealing) of the meltable adhesive material 474e.

Such a heat-sealing approach may comprise applying energy locally to melt a portion of a meltable adhesive material 474e to seal the lens chamber 401e of the eye-implantable device 400e. For example, at least a portion of the first 410e and/or second 412e chamber portion(s) proximate the first 420e and/or second 422e mating surface may comprise a meltable adhesive material 474e. During assembly, the meltable adhesive material 474e could be melted (e.g., by application of heat using a laser) to mechanically couple the first chamber portion 410e and the second chamber portion 412e when the first mating surface 420e is in contact with the second mating surface 422e. In some examples, a high-precision heating element (e.g., a laser) is used so that the energy can be localized to the region of the meltable adhesive material 474e. Additionally or alternatively, an opaque substrate or other material (e.g., elastic nitinol, an evaporated thin metal film) could be disposed underneath or adjacent to the meltable adhesive material to absorb the laser radiation and convert it locally to heat. In some examples, the meltable adhesive material 474e may differ from the material of the eye-implantable device 400e (i.e., the material of the first 410e and/or second 412e chamber portions). For instance, the meltable adhesive material 474e may have a lower melting point than the material of the eye-implantable device 400e to facilitate melting of the meltable region 474e, prevent melting of the first 410e and/or second 412e chamber portions, to avoid damaging or otherwise impeding operation of the eye and/or eye-implantable device 400e, or to provide some other benefit. In some embodiments, the melting temperature of the meltable adhesive material 474e is greater than body temperature, e.g., in the range of 40-60 degrees Celsius.

### IV. EXAMPLE ELECTRONICS OF DEVICES

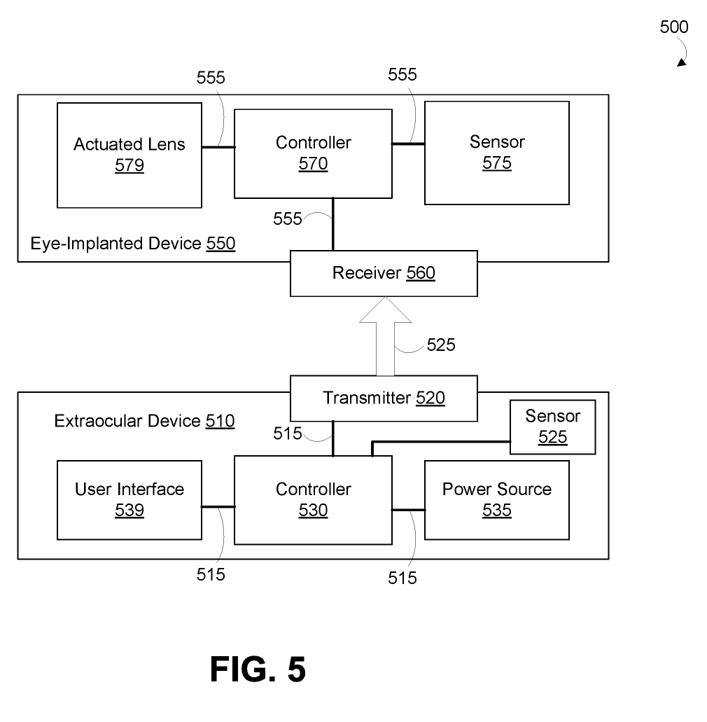

FIG. 5 is a block diagram of a system 500 that includes an extraocular device 510 wirelessly transmitting wireless signals 525 to an eye-implanted device 550. The wireless signals 525 may include wireless power signals to provide power to the eye-implanted device 550, control signals to control the operation of the eye-implanted device 550 (e.g., to control an optical power provided by an actuated lens 579 of the eye-implanted device 550), or other wireless signals. The extraocular device 510 may be a body-mounted device, e.g., a contact lens, a subconjunctival device, a head-mounted display, or some other type of head-mounted device. Additionally or alternatively, the extraocular device 510 may be a handheld device like a cell phone, a device incorporated into furniture, e.g., into a bed to facilitate charging of the eye-implantable device 550 while a user sleeps, or may take some other form(s). The eye-implanted device 550 is implanted on or within an eye of a user.

The extraocular device 510 includes a controller 530, user interface 539, a transmitter 520, a power source 535, and a sensor 525. The transmitter 520 can be operated to wirelessly transmit power, commands, or other signals to the eye-implanted device 550 in an eye. The transmitter 520, the controller 530, the power source 535, the user interface 539, and the sensor 525 can all be connected together via interconnects 515, e.g., via wires, cables and/or, patterns of metallic traces formed on a printed circuit board or other substrate material on which the components may be disposed. Further, the transmitter 520 could comprise metallic traces or patterns formed on such a substrate material (e.g., to form antennas, impedance matching elements, plates of capacitors, electrodes, mirrors or diffraction gratings).

The transmitter 520 can include light-emitting elements (e.g., LEDs, lasers, VCSELs), radio-frequency electromagnetic energy-transmitting elements (e.g., antennas, coils), elements configured to inject a time-varying current into tissues or fluids of a body (e.g., electrodes), or other elements configured to transmit, e.g., power from the power source 535 to the implanted device 550. The transmitter 520 could be configured to control an intensity, a phase, a frequency, a polarization, a direction, or some other properties of wireless signals transmitted from the transmitter 520 to indicate information. The transmitter 520 could be configured to provide power to the eye-implanted device 550 when the extraocular device 510 is not mounted to an eye or body of a user (e.g., when the user is sleeping in a bed such that the eye-implanted device 550 within an eye of the user is proximate to the extraocular device 510) or while the extraocular device 510 is mounted to the eye or body of the user.

The power source 535 may provide power to the extraocular device 510 to, e.g., wirelessly recharge a rechargeable battery of the power source 535 in embodiments wherein the extraocular device 510 is an eye-mountable device. The power source 535 could include a battery (e.g., single-use alkaline batteries, rechargeable lithium-polymer batteries), a solar cell, connection to a mains power source, or some other source of energy.

The sensor 525 may be configured to detect physiological properties (e.g., a pupillary diameter of an eye), environmental parameters (e.g., an ambient light level, a distance between eyes of a user and an object at which the user is looking), to detect movements of the eye and/or eyelids of a user (e.g., to detect a vergence of the eyes), or to otherwise detect physical parameters that may be relevant to the operation of the extraocular device 510 and/or the eye-implanted device 550. The user interface 539 may include displays, inputs, speakers, microphones, touchscreens, buttons, scroll wheels, or other elements to facilitate receiving information (e.g., commands) from a user and/or to provide information (e.g., a command interface, a battery status or other information about the devices 510, 550) to a user. For example, the user interface 539 may be operated to receive commands from a user related to a desired optical power of the eye-implanted device 550 and/or information about a distance a user wishes to see or some other information related to an optical power that could be desired from the eye-implanted device 550.

In some embodiments, some or all of the functions provided by the extraocular device 510 could be provided by a device that is implanted on or within the eye, the eye socket, or some other tissue of a person. For example, a device could be implanted beneath a sclera of an eye, within a lens capsule of an eye, within the vitreous humor of an eye, or at some other location to facilitate the device providing power, control signals, or some other functionality to the eye-implanted device 550. Such an additional device could be coupled to the eye-implanted device 550 via a tether, or could provide power and/or communicate with the eye-implanted device 550 via wireless signals. Such an additional device could be provided in order to ease volume requirements of an implanted system (e.g., to provide a larger battery by situating such battery outside a lens capsule of an eye), to provide physiological signals that are not accessible from within the lens chamber of the eye (e.g., activity of the extra-ocular muscles), to facilitate replacement of implanted components of the system 500 (e.g., to provide a method for replacing a battery of the system without explanting the actuated lens 579 or other elements of the system 500), or to provide some other benefit.

The eye-implanted device 550 includes a controller 570, a sensor 575, a receiver 560, and an actuated lens 579. The actuated lens 579 could be an electrowetting lens as described herein. The receiver 560 can be operated to receive power or other wireless signals 525 wirelessly transmitted by the transmitter 520 (e.g., from the power source 535 of the extraocular device 510). This could include receiving optical signals (e.g., via a photovoltaic cell, photodiode, or other light-sensitive elements), radio frequency electromagnetic signals (e.g., via an antenna, via a coil), an electrical current or potential in the tissues or fluids surrounding the eye-implanted device 550 (e.g., via electrodes), or receiving some other signals wirelessly transmitted from the extraocular device 510. The eye-implanted device 550 could include a capacitor, a battery, or other type of energy storage device to provide energy for use by the device 550 when power is unavailable from the other systems (e.g., when the extraocular device 510 is not mounted to or otherwise proximate to the eye-implanted device 550).

The sensor 575 is configured to detect a physiological property of the body (e.g., a pressure or force, a biopotential, a light intensity). In a particular example, the sensor 575 could be an accommodation sensor configured to detect, directly or indirectly, accommodation forces exerted on a lens capsule of the eye, e.g., by detecting a force or pressure within the lens capsule via haptics, via an elastic material disposed in the lens capsule, via detection of electrical activity of the ciliary muscles, or via some other means.

The actuated lens 579 is operable to control an optical power that is provided to the eye by the actuated lens 579. Operating the actuated lens 579 to control the optical power of the lens could include applying a voltage to a liquid crystal of the lens 579, applying a voltage to electrodes of an electrowetting actuated lens 579 or operating a pump or some other element to control a pressure and/or disposition of a fluid within the lens 579, or controlling the optical power of the lens by some other method.

The eye-implanted device 550 and/or extraocular device 510 could include additional or alternative elements, and could include more or fewer elements than those illustrated in FIG. 5. This could include the eye-implanted device 550 including elements configured to transmit wireless signals to the extraocular device 510 and the extraocular device 510 including elements configure to receive such transmitted signals. In such an example, the eye-implanted device 550 and the extraocular device 510 could additionally include a transmitter and receiver, respectively. Additionally or alternatively, the illustrated receiver 560 and transmitter 520 could be configured as transceivers to facilitate bidirectional communication and/or to share one or more elements (e.g., antennas, filters, coils, power conditioning systems) in common with other elements configured to facilitate bidirectional communication.

It is noted that the block diagram shown in FIG. 5 is described in connection with functional modules for convenience in description. However, embodiments of the extraocular device 510 and/or eye-implanted device 550 can be arranged with one or more of the functional modules (“sub-systems”) implemented in a single chip, integrated circuit, and/or physical feature. That is, the functional blocks in FIG. 5 need not be implemented as separated modules. Moreover, one or more of the functional modules described in FIG. 5 can be implemented by separately packaged chips or other components electrically connected to one another. Further, note that an extraocular device and/or an eye-implantable device as described herein could include additional or alternative components to those shown in FIG. 5 (e.g., additional sensors, actuated lenses, displays, retinal stimulator arrays, electrodes, batteries, controllers, transmitters, receivers, stimulators, etc.). For example, the power source 535 of the extraocular device 510 could be a single-use battery and the extraocular device 510 could be operated as a single-use device (e.g., operated until the battery of the power source 535 is depleted and then discarded and/or recycled).

### V. EXAMPLE METHODS

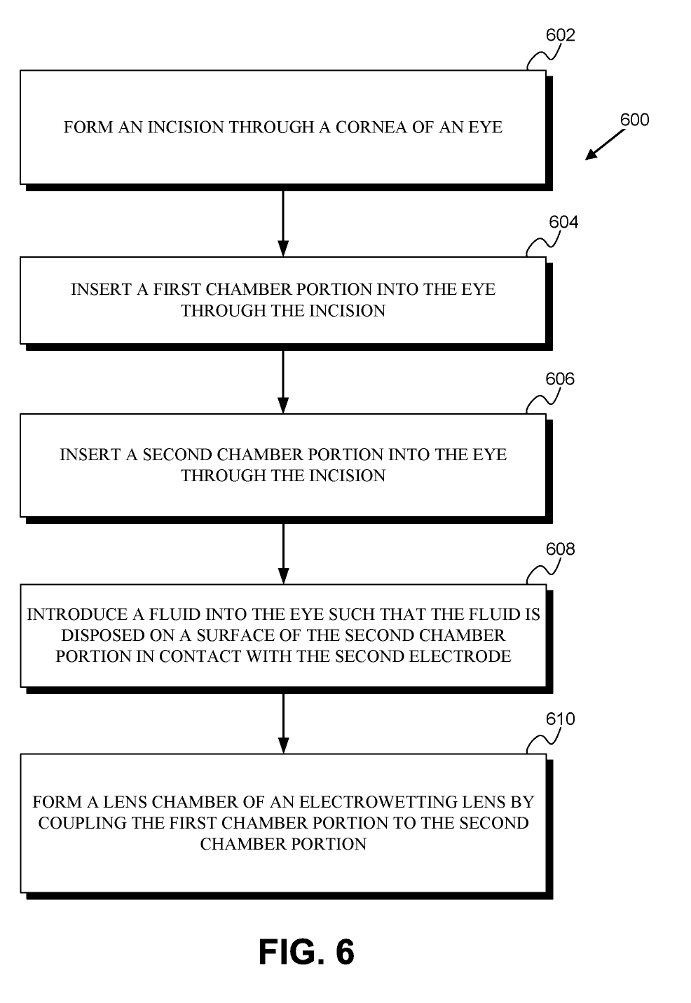

FIG. 6 is a flowchart of a method 600 for implanting an electrowetting lens and/or other elements of an eye-implantable device as described herein into a human eye. The eye-implantable device includes (i) a first chamber portion comprising a first mating surface, wherein the first chamber portion is flexible; (ii) a second chamber portion comprising a second mating surface, wherein the second mating surface is shaped to mate with the first mating surface, wherein the second chamber portion is flexible; (iii) an electrode included in at least one of the first chamber portion or second chamber portion, wherein the electrode comprises a dielectric layer; and (iv) a fluid disposed on a surface of the second chamber portion in contact with the electrode. Such an eye-implantable device could also include further electrodes on the first and/or second chamber portions, a further fluid disposed on the first and/or second chamber portions, a controller connected to the electrodes, or other components that facilitate the insertion, assembly, or operation of the eye-implantable device or provide some other benefit. Method 600 may include one or more steps, processes, and/or functions as illustrated by one or more of blocks 602 through 610. Although the blocks are illustrated in a sequential order, a number of these blocks may also be performed simultaneously and/or in a different order than those illustrated. Also, the various blocks may be combined into fewer blocks, divided into additional blocks, and/or removed based upon specific implementations.

The method 600 includes forming an incision through the cornea of an eye (602). This could include operating a scalpel, a laser, a diamond blade, a metal blade, or some other instruments to create an incision through the cornea. The incision could be created by creating multiple separate cuts or incisions into the cornea. For example, a first cut could be made perpendicular to the surface of the sclera, and one or more subsequent cuts could be made at other angles (e.g., tangential angles) relative to the sclera. The incision could be formed to be water-tight, to cause a minimum of astigmatism, or to satisfy some other considerations. The formation of the incision could be accompanied by mechanical stabilization of the eye (e.g., using fixation rings, forceps, or other means), administration of topical or global anesthesia, or some other steps. The formed incision could have a length or other dimension within some specified range; e.g., the incision could be less than 4 millimeters long, or less than 2 millimeters long.

The method 600 includes inserting the first chamber portion into the eye through the incision, wherein the first chamber portion comprises a first mating surface, and wherein the first chamber portion is flexible (604). The method 600 also includes inserting the second chamber portion into the eye through the incision, wherein the second chamber portion comprises a second mating surface that is shaped to mate with the first mating surface, wherein the second chamber portions is flexible (606). In some examples, one or both of the chamber portions could be rolled, folded, or otherwise manipulated to reduce one or more dimensions of the device (e.g., in order to facilitate insertion of the device through a smaller incision) and the method 600 could include unrolling, unfolding, positioning or otherwise manipulating the eye-implantable device subsequent to inserting the device through the incision. For example, a first chamber portion and a second chamber portion that are flexible could be inserted through the incision in a rolled or folded state. Insertion of a first and/or a second chamber portion may further comprise inserting electrodes, antennae, sensors, a controller that is electronically coupled to the electrodes, or other electronic components, fluids, or other components of an electrowetting lens. Insertion could further include using forceps or some other means to insert the first and second chamber portions through the incision. Subsequent to inserting the first and second chamber portions into the eye, they could be unrolled, unfolded, or otherwise manipulated to prepare for assembly of the electrowetting lens. Additionally or alternatively, the first or second chamber portions could include tabs, rods, or other features to facilitate such insertion. Such features could be later removed from the chamber portions (e.g., by cutting, crimping, laser cutting, or some other means) or could remain as part of the eye-implantable device following implantation.

The method 600 further includes introducing a fluid into the eye such that the fluid is disposed on a surface of the second chamber portion in contact with the second electrode (608). Such a fluid may be introduced into and/or otherwise disposed on a surface of the second chamber portion in a variety of ways. For example, the fluid may be introduced into an environment of the first chamber portion (e.g., into a lens capsule, a posterior chamber, or some other environment containing the first chamber portion) prior to mating the second chamber portion to the first chamber portion. In such an example, coupling the first chamber portion and the second chamber portion together to form the electrowetting lens causes the fluid (e.g., an amount of a saline fluid introduced into the environment of the first chamber portion), to be disposed on a surface of the second chamber portion in contact with the second electrode. Additionally or alternatively, the fluid may be introduced into the eye after the lens chamber of the eye-implantable has been formed. The addition of one or more fluids to the lens chamber and/or the second chamber portion thereof could be achieved by penetrating the lens chamber or a septum or other element of the electrowetting lens in fluid communication with the lens chamber with a needle. The fluid(s) may then be disposed on a surface of the second chamber portion via the needle. In another example, the fluid(s) could be introduced via a tube or duct that is formed from or otherwise attached to the electrowetting lens. Means for adding fluid(s) to the electrowetting lens could also be used to remove an amount of fluid from the electrowetting lens (e.g., to control a volume of the lens chamber, to allow rinsing, de-bubbling, or de-gassing of fluid(s) within the lens chamber, to allow replacement of a fluid disposed within the lens chamber with an alternative fluid, or to provide some other benefit). The method 600 further includes forming a lens chamber of the electrowetting lens by coupling the first chamber portion to the second chamber portion, wherein coupling the first chamber portion to the second chamber portion comprises mating the first mating surface with the second mating surface (610). For example, the first and second chamber portions could include a first and second mating surface, respectively. Coupling the first and second chamber portions could include bringing the first mating surface in contact with the second mating surface such that respective chamber surfaces of the first and second chamber portions form respective internal surfaces of the formed lens chamber. of the electrowetting lens.

Mating of the first and second chamber portion as described herein could further include applying a sealant to the first and/or second chamber portion and curing the sealant such that the sealant inhibits fluid flow between the lens chamber and an external environment of the eye-implantable device. In some embodiments, a heat-sealing approach is used, wherein at least a portion of the first or second chamber portions comprises a meltable adhesive material, and energy is used to melt a portion of the meltable adhesive material to couple the first and second chamber portions. Additionally or alternatively, the first and second chamber portions may be mated via a snap fit. For example, the first and second chamber portions may comprise a first and second clamping feature, respectively, which mechanically couple the first and second chamber portions when the first mating surface is in contact with the second mating surface. The first and second chamber portions may further comprise a first and second material with a first and second modulus of elasticity, respectively, and coupling the first chamber portion to the second chamber portion could comprise elastically deforming at least one of the first or second materials such that a force is exerted between the first clamping feature and the second clamping feature. Additionally or alternatively, a clamp could be used to couple the first and second chamber portions. In such an example, the first and second chamber portions comprise a first and second clamping surface, which correspond to a third and fourth clamping surface, respectively, located on the clamp. Such a clamp could be used to couple the first and second chamber portions by putting the first clamping surface in contact with the third clamping surface and the second clamping surface in contact with the fourth clamping surface such that the clamp applies forces to the first clamping surface and the second clamping surface. Alternative means of coupling the first and second chamber portions are also considered.

Method 600 could include additional steps or elements in addition to those depicted in FIG. 6. For example, the method 600 could include adding or removing other materials or fluids to or from a lens chamber of an eye-implantable device, e.g., adding or removing an amount of a further fluid. This could be performed using one or more needles, one or more tubes connected between the device and external systems, or via some other method. For example, the lens chamber could be rinsed by introducing an amount of the first fluid into the lens chamber, e.g., via the needle, via one or more further needles, via the tube, via one or more further tubes, or via some other means. Additionally or alternatively, an amount of the first fluid could be removed from the lens chamber, e.g., by applying suction via the needle, tube, or other fluid transfer means. The method 600 could include removing gases from one or both of the first or second fluids prior to introducing such fluids into the lens chamber.

The method 600 could include retracting the needle, crimping the tube, retracting a sectioned portion of the tube, and/or performing some further assembly or other processes to an eye-implantable device. For example, the eye-implantable device could include a tube that protrudes from the device and that provides a fluid pathway into the lens chamber (e.g., to facilitate the exit of an amount of the first fluid from the lens chamber when the second fluid is introduced). The method 600 could include crimping, cutting, or otherwise manipulating such a tube, e.g., to inhibit fluid flow through the tube. Crimping such a tube could include applying a mechanical, acoustical, electromagnetic, or thermal force to induce some change in the material of the tube, e.g., to melt the tube, to cause opposite walls of the tube to chemically interact, to cause a material in the tube to undergo photopolymerization, or to cause some other process. Additionally or alternatively, a staple or other means could be applied to the tube to crimp the tube.

The method 600 could include further surgical manipulations of the eye, e.g., the formation of a hole in the lens capsule and/or the removal of the crystalline lens, the removal of a previously implanted device (e.g., a static IOL). In some examples, the eye-implantable device could be implanted through the sclera or via some other route, and the methods 600, 700 could include forming alternative incisions (e.g., through the sclera) and inserting the device through such alternative incisions. The method 600 could further include washing, irrigation and/or debubbling of a region within an eye (e.g., the lens capsule of the eye) to remove debris, clear excess viscoelastic gels, improve the optical clarity of the assembled device, or to provide some other benefit. Verification testing could also be performed to determine whether the assembled device is functional, to adjust or program individual components, or to calibrate the device (e.g., to determine the optical power as a function of voltage applied). For example, this could include using the controller to apply a voltage between the electrodes to assess the functionality and optical performance of the assembled electrowetting lens. Other methods of in situ assembly, manipulation, and/or testing of eye-implantable device components as described herein are contemplated.

### VI. CONCLUSION

Where example embodiments involve information related to a person or a device of a person, the embodiments should be understood to include privacy controls. Such privacy controls include, at least, anonymization of device identifiers, transparency and user controls, including functionality that would enable users to modify or delete information relating to the user's use of a product.

Further, in situations in where embodiments discussed herein collect personal information about users, or may make use of personal information, the users may be provided with an opportunity to control whether programs or features collect user information (e.g., information about a user's medical history, social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

The particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

Additionally, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are contemplated herein.

What is claimed is:

1. A method comprising:
forming an incision through a cornea of an eye;
inserting a first chamber portion into the eye through the incision, wherein the first chamber portion comprises a first mating surface, and wherein the first chamber portion is flexible;
inserting a second chamber portion into the eye through the incision, wherein the second chamber portion comprises a second mating surface that is shaped to mate with the first mating surface, wherein the second chamber portion is flexible, wherein the second chamber portion comprises an electrode, and wherein the electrode comprises a dielectric layer;
introducing a fluid into the eye such that the fluid is disposed on a surface of the second chamber portion in contact with the second electrode; and
forming a lens chamber of an electrowetting lens by coupling the first chamber portion to the second chamber portion, wherein coupling the first chamber portion to the second chamber portion comprises mating the first mating surface with the second mating surface.

2. The method of claim 1, wherein inserting the second chamber portion into the eye through the incision comprises inserting the second chamber portion in a folded state, and further comprising:
subsequent to inserting the second chamber portion into the eye through the incision, unfolding the second chamber portion.

3. A kit of components for assembling an eye-implantable device, the kit comprising:
a first chamber portion comprising a first mating surface, wherein the first chamber portion is flexible;
a second chamber portion comprising a second mating surface, wherein the second mating surface is shaped to mate with the first mating surface, wherein the second chamber portion is flexible;
wherein the first chamber portion and the second chamber portion form a lens chamber of an electrowetting lens when the first mating surface is mated with the second mating surface; and
a fluid packaged for injection into an eye onto a surface of at least one of the first chamber portion or the second chamber portion.

4. The components of claim 3, wherein the second chamber portion further comprises:
a first electrode;
a second electrode; and
a controller electronically coupled to the first electrode and the second electrode, wherein the controller is configured to apply a voltage between the first electrode and the second electrode.

5. The components of claim 3, further comprising:
a first electrode;
a second electrode included in the second chamber portion; and
a controller for applying a voltage between the first electrode and the second electrode, wherein the controller is included in the second chamber portion electronically coupled to the second electrode, and wherein the controller is electronically coupled to the first electrode when the first mating surface is mated with the second mating surface.

6. The components of claim 4, further comprising a tether, wherein the controller is coupled to the second chamber portion via the tether.

7. The components of claim 3, wherein a pressure-sensitive adhesive is disposed on at least one of the first mating surface or the second mating surface.

8. The components of claim 3, wherein the first chamber portion further comprises a first clamping feature, wherein the second chamber portion further comprises a second clamping feature, and wherein the first chamber portion and the second chamber portion are mechanically coupled via the first clamping feature and the second clamping feature when the first mating surface is mated with the second mating surface.

9. The components of claim 8, wherein the first chamber portion comprises a first material having a first modulus of elasticity, wherein the second chamber portion comprises a second material having a second modulus of elasticity, and wherein the first modulus of elasticity differs from the second modulus of elasticity such that, when the first mating surface is mated with the second mating surface, a force is exerted between the first clamping feature and the second clamping feature.

10. The components of claim 3, further comprising a clamp, wherein the first chamber portion comprises a first clamping surface, wherein the second chamber portion comprises a second clamping surface, wherein the clamp comprises a third clamping surface shaped to interact with the first clamping surface, wherein the clamp further comprises a fourth clamping surface shaped to interact with the second clamping surface, and wherein the clamp applies forces to the first clamping surface and the second clamping surface when the first mating surface is mated with the second mating surface, the first clamping surface is in contact with the third clamping surface, and the second clamping surface is in contact with the fourth clamping surface.

11. The components of claim 3, wherein at least one of the first mating surface or the second mating surface comprises a meltable adhesive material.

12. The components of claim 3, wherein the second chamber portion is in at least one of a rolled state or a folded state.

13. A component for an eye-implantable device comprising:
a chamber portion comprising a concave lens chamber surface, wherein the chamber portion is flexible;
a first electrode disposed on the concave lens chamber surface;
a second electrode disposed on the concave lens chamber surface, wherein the second electrode comprises a dielectric coating; and
a controller, wherein the controller is electronically coupled to the first electrode and the second electrode, and wherein the controller is configured to apply a voltage between the first electrode and the second electrode.

14. The component of claim 13, wherein the chamber portion comprises a hydrophobic coating on at least a portion of the concave lens chamber surface.

15. The component of claim 13, further comprising a tether, wherein the controller is coupled to the chamber portion via the tether.

16. The component of claim 13, wherein the chamber portion is configured in at least one of a rolled state or a folded state.

17. The component of claim 13, wherein the chamber portion further comprises a mating surface, wherein a pressure-sensitive adhesive is disposed on at least a portion of the mating surface.

18. The component of claim 13, wherein at least a portion of the chamber portion comprises a meltable adhesive material.

19. The component of claim 13, wherein the chamber portion comprises a convex optical surface, wherein a shape of the convex optical surface is specified to provide an optical power.

20. The component of claim 13, wherein the chamber portion comprises a polymeric material that comprises 2-phenylethyl acrylate units and 2-phenylethyl methacrylate units.
